(19) United States
(12) Patent Application Publication
(10) Pub. No.: US 2019/0203253 A1
(43) Pub. Date: Jul. 4, 2019

(54) METHODS OF IDENTIFYING AND TREATING CANCER PATIENTS WITH AN EPHB6 DEFICIENCY

(71) Applicants: University of Saskatchewan, Saskatoon (CA); SASKATCHEWAN CANCER AGENCY, Saskatchewan (CA)

(72) Inventors: Franco Joseph Vizeacoumar, Saskatoon (CA); Andrew Freywald, Saskatoon (CA); James Mathew Paul, Medicine Hat (CA); Frederick Sagayaraj Vizeacoumar, Saskatoon (CA)

(21) Appl. No.: 16/312,410

(22) PCT Filed: Jul. 5, 2017
(86) PCT No.: PCT/CA2017/050812

§ 371 Date: Dec. 21, 2018

Related U.S. Application Data
(60) Provisional application No. 62/358,393, filed on Jul. 5, 2016.

(51) Int. Cl. C12Q 1/02; A61P 35/00; A61K 31/506; C12Q 1/686

(52) U.S. Cl. CPC C12Q 1/025; A61P 35/00; C40B 30/06; C12Q 1/686; A61K 31/506

(57) ABSTRACT

Methods for identifying a subject with a cancer eligible for treatment with an inhibitor of a Table 1 molecule, optionally a SRC kinase inhibitor or a MET kinase inhibitor, are provided. The methods comprise testing a biological sample from the subject for a deficiency in EPHB6 receptor levels, wherein the subject is eligible for treatment with an inhibitor of a Table 1 molecule, optionally a SRC kinase inhibitor or a MET kinase inhibitor, if EPHB6 receptor levels in the biological sample are deficient.

### CROSS-REFERENCE TO RELATED APPLICATION

This is a Patent Cooperation Treater which claims the benefit of 35 U.S.C. 119 based on the priority of U.S. Provisional Patent Application No, 62/358,393, filed Jul 5, 2016, which is incorporated herein by reference in its entirety.

### FIELD

The disclosure pertains to methods for identifying patients for treatment and treating patients with a EPHB6 receptor deficiency and more particularly to identifying patients that are deficient for EPHB6 receptor for treating with an inhibitor of a Table 1 molecule, for example a SRC kinase inhibitor or a MET kinase inhibitor.

### BACKGROUND

The establishment of the estrogen receptor and human epidermal growth factor receptor-2 (HER2) as therapeutically relevant targets marked the development of genotype-directed treatment for breast cancer patients. The initial success in inhibiting key oncogenic drivers has stimulated extensive tumor genome sequencing aiming to identify genetic alterations for developing novel personalized therapies [1]. These personalized therapies targeting oncogenic alterations within a specific tumor are associated with minimal non-specific toxicity in cancer patients. Interestingly, tumor genome sequencing has also revealed numerous non-druggable genetic alterations such as deep deletions or epigenetic silencing in cancer cells. Development of mechanisms or tools to efficiently utilize these loss-of-function alterations for therapeutic purposes would dramatically expand our options in treatment personalization. In this context, the identification of synthetic lethal (SL) interactions, where suppression of one gene causes lethality only when another gene is also inactivated [2, 3], provides a unique opportunity to target these loss-of-function genetic defects.

The EPHB6 receptor is a member of the Eph group that lacks catalytic activity due to several intrinsic alterations in the sequence of its kinase domain [4] and in contrast to other Eph receptors [5-7], EPHB6 is often downregulated in various malignancies, including metastatic lung cancer [8], melanoma [9], prostate cancer [10], ovarian carcinoma [11], gastric cancer [12], aggressive neuroblastoma [13, 14], and invasive breast cancer cell lines [15, 16]. EPHB6 has been reported to suppress metastasis in non-small cell lung cancer [17] and melanoma [18], and to actively reduce breast cancer invasiveness [19]. EPHB6 receptor deficiency may potentially be targeted by using the SL approach to further personalize cancer therapy and improve treatment in multiple malignancies.

### SUMMARY

EPHB6 is downregulated in multiple malignancies. A number of genes show synthetic lethality with EPHB6 as demonstrated in the Examples. Drugs that target one or more of the proteins encoded by these genes may be useful for treating an individual with EPHB6-deficient tumors. Disclosed herein are methods for personalizing cancer treatment.

In an aspect, there is provided a method of identifying a subject with a cancer eligible for treatment with an inhibitor of a Table 1 molecule, optionally a SRC kinase inhibitor or a MET kinase inhibitor, comprising testing a biological sample from the subject for a deficiency in EPHB6 receptor levels, wherein the subject is eligible for treatment with the inhibitor of a Table 1 molecule, optionally the SRC kinase inhibitor or the MET kinase inhibitor, if EPHB6 receptor levels in the biological sample are deficient.

In another aspect, there is provided method of treating a cancer in a subject comprising: administering an effective amount of an inhibitor of a Table 1 molecule, optionally a SRC kinase inhibitor or a MET kinase inhibitor, to a subject in need of such a treatment, wherein the subject in need of such treatment is identified by evaluating EPHB6 receptor levels in a biological sample of a subject suspected from having from cancer, having cancer or being prone to having cancer, and wherein a deficiency in EPHB6 receptor levels in the biological sample, optionally compared to a control, indicates responsiveness of the subject to the inhibitor of a Table 1 molecule, optionally the SRC kinase inhibitor or the MET kinase inhibitor.

Another aspect is a method of treating a cancer in a patient, comprising testing for a deficiency in EPHB6 receptor levels in a biological sample from the patient and administering a therapeutically effective amount of an inhibitor of a Table 1 molecule, optionally a SRC kinase inhibitor or a MET kinase inhibitor, to the patient if the biological sample tests positive for a deficiency in EPHB6 receptor levels.

Also provided in another aspect is a method of personalizing treatment in a subject having or suspected of having cancer comprising measuring EPHB6 receptor levels in a biological sample obtained from the subject, optionally comparing the measured EPHB6 receptor levels to a control, treating the subject with an inhibitor of a Table 1 molecule, optionally a SRC kinase inhibitor or a MET kinase inhibitor when the EPHB6 receptor levels are deficient, and otherwise treating the subject with an alternate treatment, for example when the EPHB6 receptor levels are comparable or increased compared to a control such as adjacent normal tissue.

A further aspect includes a method of selecting a therapeutic for a subject having or suspected of having cancer, the method comprising:
a) obtaining a biological sample from the subject,
b) measuring EPHB6 receptor levels in the biological sample, and
c) selecting an inhibitor of a Table 1 molecule, optionally a SRC kinase inhibitor or a MET kinase inhibitor, as the therapeutic when a deficiency in EPHB6 receptor levels is measured in the biological sample or selecting an alternate therapeutic, for example when the EPHB6 receptor levels are comparable or increased compared to a control such as adjacent normal tissue.

In a further aspect, there is provided a use of an inhibitor of a Table 1 molecule, optionally a SRC kinase inhibitor or a MET kinase inhibitor, for treating a subject in need thereof, wherein the subject in need thereof is identified by evaluating EPHB6 receptor levels in a biological sample of a subject suspected of having cancer, having cancer of being prone to having cancer, and a deficiency in EPHB6 receptor levels in the biological sample, optionally compared to a control, identifies the subjects as responsive to the inhibitor of a Table 1 molecule, optionally the SRC kinase inhibitor or the MET kinase inhibitor.

Also provided in another aspect is an inhibitor of a Table 1 molecule, optionally a SRC kinase inhibitor or a MET kinase inhibitor, for use in treating a subject in need thereof, wherein the subject in need thereof is identified by evaluating EPHB6 receptor levels in a biological sample of a subject suspected of having cancer, having cancer of being prone to having cancer, and wherein a deficiency in EPHB6 receptor levels in the biological sample, optionally compared to a control, identifies the subject as responsive to the inhibitor of a Table 1 molecule, optionally the SRC kinase inhibitor or the MET kinase inhibitor.

Another aspect is a screening assay, comprising:
contacting a control cancer cell sample with a test candidate;
contacting a test cancer cell sample deficient in EPHB6 receptor levels with the test candidate;
measuring an effect of the test candidate on the control cancer cell sample and on the test cancer cell sample;
comparing the effect of the test candidate on the control cancer cell sample and on the test cancer cell sample; and
identifying the test candidate as a putative inhibitor, optionally a putative SRC kinase inhibitor or a putative MET kinase inhibitor, when the effect measured is greater on the test cancer cell sample compared to the control cancer cell sample.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1. EPHB6 is downregulated in multiple human malignancies. A. EPHB6 expression was analyzed in twenty-three different cancer types and matching normal tissue controls using data from The Cancer Genome Atlas (TCGA). The number of samples analyzed is shown on the x-axis. B. Analysis of EPHB6 promoter methylation in eighteen different cancer types and matching normal tissue controls using data from TCGA. The number of samples analyzed is shown on the x-axis. The best methylation site was taken following the pre-processing step as outlined by the Broad Institute. The legend for panels (A) and (B) is presented below panel (B). C. EPHB6 expression in normal and triple negative breast cancer (TNBC) samples. TNBC samples were identified in the TCGA dataset based on the immunohistochemistry test. Statistical significance was computed using the Mann-Whitney U test.

FIG. 2. Genome-wide SL screen of EPHB6. A. EPHB6 expression in EPHB6-deficient triple-negative breast cancer cells, MDA-MB-231, stably transfected with the pcDNA3 expression vector encoding wild-type EPHB6 (MDA-B6), myc-tagged EPHB6 (MDA-B6-M), or mock-transfected with empty pcDNA3 (MDA-pc3) was examined by Western blotting with anti-EPHB6. Western blotting with anti-tubulin was used as a loading control. B. MDA-pc3, MDA-B6, and MDA-B6-M cells were stained with anti-EPHB6 and a FITC-conjugated secondary antibody, and analyzed by flow cytometry. Matching non-specific IgG was used as a control (Control IgG). C. Schematic showing the steps of the shRNA pooled screening pipeline. D. Pearson correlation between replicates of the pooled screen are clustered using hierarchical clustering with complete linkage. E. Precision (TP/(TP+FP)) recall (TP/(TP+FN)) curve measuring the core essential and non-essential genes from the EPHB6 pooled screen. F. Scatter plot showing the DCC score for every gene when MDA-pc3 is compared to both MDA-B6 and MDA-B6-M. G. Analysis showing Gene Ontology terms associated with each screen. H. Expected cellular distribution of EPHB6 synthetic lethal partners according to the Compartments Subcellular Localization Database.

FIG. 3. SRC is identified as a SL interacting partner of EPHB6. A. Correlation clustergram showing expression of synthetic lethal hits (vertical) relative to EPHB6 expression (horizontal) across human malignancies. B. Expression analysis of SRC in twenty-four different cancer types and normal tissue controls using data from TCGA. The number of samples analyzed is shown on the x-axis. C. MDA-pc3 and MDA-B6 cells were transduced with SRC-targeting shRNA or luciferase-targeting shRNA as a control, and cultured in 96-well plates for 96 hours after puromycin selection. Cells were stained with resazurin and fluorescence was measured using a SpectraMax M5 microplate reader to determine cell suppression. The graph represents percentage of cell suppression by SRC hairpin relative to matching luciferase hairpin controls. Five wells were analyzed per condition. Experiment was performed three times. *, P<0.05, Student's t-test. D. Schematic representation of the CRISPR/Cas9 strategy to validate the SL interaction. Cells of interest are stably transduced with a construct encoding src-targeting sgRNA and blue fluorescent protein (BFP), followed by the selection in the presence of 2 μg/mL of puromycin. The selected cells are transiently transfected with a construct encoding Cas9-2A-GFP. E. MDA-pc3 and MDA-B6 cells were stably transduced with the src-targeting sgRNA construct that also encoded the blue fluorescent protein (BFP) and selected in the presence of 2 μg/ml of puromycin. The selected cells were transiently transfected with Cas9-GFP in 96-well plates and consistent transfection efficiency was confirmed by quantifying cells with green and blue fluorescence using the ImageXpress Micro XLS widefield automated fluorescence microscope and the MetaXpress version 6 software. The graph represents percentage of cells co-expressing Cas9-GFP and BFP relative to total cell numbers per well. F. Surviving transfected cells from (E) were quantified at the indicated time points with the ImageXpress Micro XLS microscope and the MetaXpress software. The graph represents survival of transfected cells as a percentage relative to numbers of matching control cells expressing sgRNA/BFP only. Normalization on control cells was performed to account for a potential difference in proliferation rates of MDA-pc3 and MDA-B6 cells. In (E) and (F) each graph represents two independent experiments. At least ten wells were analyzed per condition in each experiment. *, P<0.05, Student's t-test. n.s., statistically not significant. G. PCR amplification of src-sgRNA targeted genomic regions (with and without Cas9 expression) and DNA cleavage by the Detection Enzyme (GeneArt Genomic cleavage detection kit) are shown to demonstrate knockout of src.

FIG. 4. SL interaction between EPHB6 and SRC. A. MDA-pc3 and MDA-B6 cells were cultured in 96-well plates with the indicated concentrations of SU6656 or matching volumes of DMSO for 72 h. Cells were stained with resazurin and fluorescence was measured using a SpectraMax M5 plate reader to determine cell suppression. Five wells were analyzed per condition. The graph shows percentage of cell inhibition relative to DMSO control. B. MDA-pc3 and MDA-B6 cells were cultured in 96-well plates with the indicated concentrations of KX2-391 or matching volumes of DMSO for 72 h. Cells were stained with resazurin and fluorescence was measured using a SpectraMax M5 plate reader to determine cell suppression. Five wells were analyzed per condition. The graph shows percentage of cell inhibition relative to DMSO control. C. MDA-B6 and MDA-pc3 cells were transduced with lentivirus encoding pLD-GFP-Puro or pLD-RFP-Puro as indicated. Cells were selected with 2 μg/mL puromycin for 48 h, cultured in monolayer, and imaged by confocal microscopy at 100× magnification. D. GFP-expressing MDA-B6 cells (MDA-B6-GFP) and RFP-expressing MDA-pc3 (MDA-pc3-RFP) were combined in equal numbers at the indicated cell densities in 24-well plates, and cultured with 25 nM KX2-391 or DMSO for 72 h. Cells were collected and analyzed by flow cytometry and the FlowJo software. The graph represents analysis of triplicates and shows ratios of proportional representation of KX2-391-treated fluorescent populations relative to matching DMSO controls. E. RFP-expressing MDA-B6 (MDA-B6-RFP) and GFP-expressing MDA-pc3 (MDA-pc3-GFP) cells were combined in equal numbers at the indicated cell densities in 24-well plates and cultured with 25 nM KX2-391 or matching volume of DMSO for 72 h. Cells were collected and analyzed by flow cytometry and the FlowJo software. The graph represents analysis of triplicates and shows ratios of proportional representation of KX2-391-treated fluorescent populations relative to matching DMSO controls. All experiments were performed at least three times. Scale bar, 100 pM. *, P<0.05, Student's t-test.

FIG. 5. Inhibition of SRC induces cell death more efficiently in EPHB6-deficient cells. A. MDA-pc3, and MDA-B6 cells were cultured in glass-bottom plates in the presence of 25 nM KX2-391 or DMSO for 72 h and stained with 2.7 μg/mL propidium iodide (PI) in phenol red-free medium. Stained cells were imaged at 200× magnification using Zeiss LSM 700 confocal microscope and PI-stained cells were counted in at least 10 randomly captured frames. Counts of PI-positive cells were normalized on the total cell numbers in matching frames. The graph shows the ratio of PI-positive cells in KX2-391-treated populations relative to matching DMSO controls. B. MDA-pc3 and MDA-B6 cells were cultured in 6-well plates in the presence of 25 nM KX2-391 or a matching volume of DMSO for 72 h. Cells were collected and stained for 7-AAD for 15 minutes at room temperature. Cells were analyzed by flow cytometry and the FlowJo software. The graph represents analysis of triplicates and shows fold change in mean fluorescence intensity in KX2-391-treated cells relative to matching DMSO controls. All experiments were performed at least three times. Scale bar, 100 μm. *, P<0.05, Student's t-test.

FIG. 6. SL relation between EPHB6 and SRC in BT-20 TNBC cells. A. Triple negative breast cancer cells, BT-20, were transduced with EPHB6-targeting shRNA (BT20-66-shRNA), or non-silencing shRNA (BT2O-NS). EPHB6 expression was analyzed by Western blotting with anti-EPHB6. Western blotting with anti-tubulin was used as a loading control. B. BT20-66-shRNA and BT2O-NS cells were cultured in 96-well plates with indicated concentrations of SU6656 or matching volumes of DMSO for 96 h. Cells were stained with resazurin and fluorescence was measured using a SpectraMax M5 plate reader to determine cell suppression. Five wells were analyzed per condition. The graph shows percentage of cell suppression relative to DMSO control. C. Cells were cultured in 96-well plates with indicated concentrations of KX2-391 or matching volumes of DMSO for 96 h. Cells were stained with resazurin and fluorescence was measured using a SpectraMax M5 plate reader to determine cell suppression. Five wells were analyzed per condition. The graph shows percentage of cell suppression relative to DMSO control. D. BT20-66-shRNA and BT2O-NS were cultured in glass-bottom plates in the presence of 35 nM KX2-391 or DMSO for 96 h. Cells were stained with 2.7 μg/mL propidium iodide (PI) in phenol red-free medium and imaged using confocal microscopy. PI-stained cells were counted in at least 10 randomly captured frames. Counts of PI-positive cells were normalized on the total cell numbers in matching frames. The graph shows the ratio of PI-positive cells in KX2-391-treated populations relative to matching DMSO controls. E. Cells were cultured in 6-well plates in the presence of 25 nM KX2-391 or DMSO for 96 h, collected and stained with 7-AAD. Stained cells were analyzed by flow cytometry and the FlowJo software. The graph represents analysis of triplicates and shows the mean fluorescence intensity of KX2-391-treated cells relative to DMSO control. All experiments were performed at least three times. Scale bar, 100 μM. *, P<0.05, Student's t-test.

FIG. 7. Analysis of the SL interaction between EPHB6 and SRC in TNBC cells and murine xenografts. A. MDA-pc3 and MDA-B6 cells were injected into the mammary fat pad region of 4-6 weeks old NOD-SCID mice (1×10⁶ cells per mouse). Mice with detectable tumors were treated twice per day with 5 mg/kg KX2-391 in DMSO/water solvent or solvent alone by oral feeding (at least 6 animals per each experimental condition). Tumor size was measured every 3 days and tumor volume was calculated with the equation: A/2*B², where A was long and B was short diameter of the tumor. The reduction in tumor growth in KX2-391-treated mice is presented as a percentage relative to matching solvent controls. The graph summarizes two independent experiments. Day 0 indicates the beginning of treatment with KX2-391 or matching solvent control. The experiments were terminated upon tumor ulceration according to the guidelines established by the Animal Research Ethics Board, University of Saskatchewan. B. KX2-391-treated MDA-B6 and MDA-pc3 tumors from (A) were extracted upon experiment termination, fixed in 10% neutral-buffered formalin, and paraffin embedded. Tumor sections were processed for immunohistochemical staining with anti-CD34 or stained with haematoxylin and eosin (H&E). Four representative fields (at 3, 6, 9, and 12 o'clock) per each stained tumor section (one for each extracted tumor) were imaged at 100× magnification and the blood vessel density per each field was analyzed with the Image-Pro Premier software. The graph represents percentage of anti-CD34-positive area relative to the overall field of view. Images of representative areas highlighted by rectangles are shown at 400× magnification. Arrows indicate representative examples of anti-CD34-stained blood vessels. At least 6 stained sections per each experimental condition representing independent tumors were used for the analysis. Scale bar, 500 μM. *, P<0.05; **, P<0.01, Student's t-test. n.s., statistically not significant.

FIG. 8. Characterization of SL interactions of EPHB6. A. Frequency chart of MDA-B6 DCC scores with P-values below 0.05 highlighted in gray. B. Frequency chart of MDA-B6-M DCC scores with P-values below 0.05 highlighted in gray. C. Network generated from the STRING database based on the function interactions of the genes. D. SRC expression in MDA-pc3 and MDAB6 cells transduced with SRC-targeting shRNA (sh149), or non-silencing shLuciferase (shLuc). SRC expression was analyzed by Western blotting with anti-SRC and quantitated by densitometry. SRC quantifications were normalized on matching tubulin controls and presented in arbitrary units (AU).

FIG. 9. Analysis of EPHB6-SRC SL interaction. A. MDA-pc3 and MDA-B6 cells were stably transduced with a src-targeting sgRNA construct that also encoded the blue fluorescent protein (BFP) and selected in the presence of 2 μg/ml of puromycin. The selected cells were transiently transfected with Cas9-GFP in 96-well plates. Green and blue fluorescence was quantified using the ImageXpress Micro XLS widefield automated fluorescence microscope and the MetaXpress version 6 software. The figure shows representative images of MDA-pc3 and MDA-B6 cells at consistent locations over the period of six days following Cas9 transfection. White highlighted cells represent those expressing BFP, while gray-highlighted cells represent those coexpressing BFP and GFP, according to the standard MetaXpress software settings. Scale bar, 250 μM. B. MDA-pc3 and MDA-B6 cells were serum-starved for 24 hours and then treated with 20 μM SU6656 or matching DMSO control for 40 minutes in the presence of 10% FBS. Cells were lysed and immunoprecipitations were performed with anti-SRC. Immunoprecipitates were resolved by SDSPAGE, transferred to the nitrocellulose membrane and Western blotted with anti-phospho-SRC (antip-SRC), recognizing SRC molecules phosphorylated on the activating tyrosine residue. The presence of SRC in matching cell lysates was monitored by Western blotting with anti-SRC.

FIG. 10. Analysis of EPHB6-MET SL interaction. A and B, MDA-B6 and MDA-pc3 (A) or BT2O-NS and BT20-shB6 (B) cells were treated for 24 hours with the indicated concentrations of MET inhibitor, ARQ197, or with matching concentrations of DMSO, as a control. Treated cells were stained with Resazurin for 2 h at 37° C. and cell survival was measured using a microplate reader. Data represent the analysis of triplicates and are shown as a percentage relative to matching DMSO controls. (*) Statistical analyses: Student's t test, P<0.05 for indicated points. All analyses represent one of at least three independent experiments.

FIG. 11. MET inhibition preferentially suppresses EPHB6-deficient cells. Hygro-selected combinations of MDA-B6-GFP and MDA-pc3-RFP (A and B) or MDA-B6-RFP and MDA-pc3-GFP (C and D) cells were seeded in 1:1 ratio and treated with 0.3 μM concentration of Met receptor inhibitor, ARQ197, or with a matching volume of DMSO, as a solvent control for 24, 32, and 48 hours. Treated cells were analyzed by flow cytometry. Bar graphs are based on the analyses of triplicates and represent a suppression of ARQ197-treated cell populations as a percentage relative to matching DMSO controls. (*) Statistical analyses: Student's t test, P<0.05 for indicated points. All analyses represent one of at least three independent experiments. The skilled person in the art will understand that the drawings, described herein, are for illustration purposes only. The drawings are not intended to limit the scope of the applicants' teachings in any way.

### DETAILED DESCRIPTION OF THE DISCLOSURE

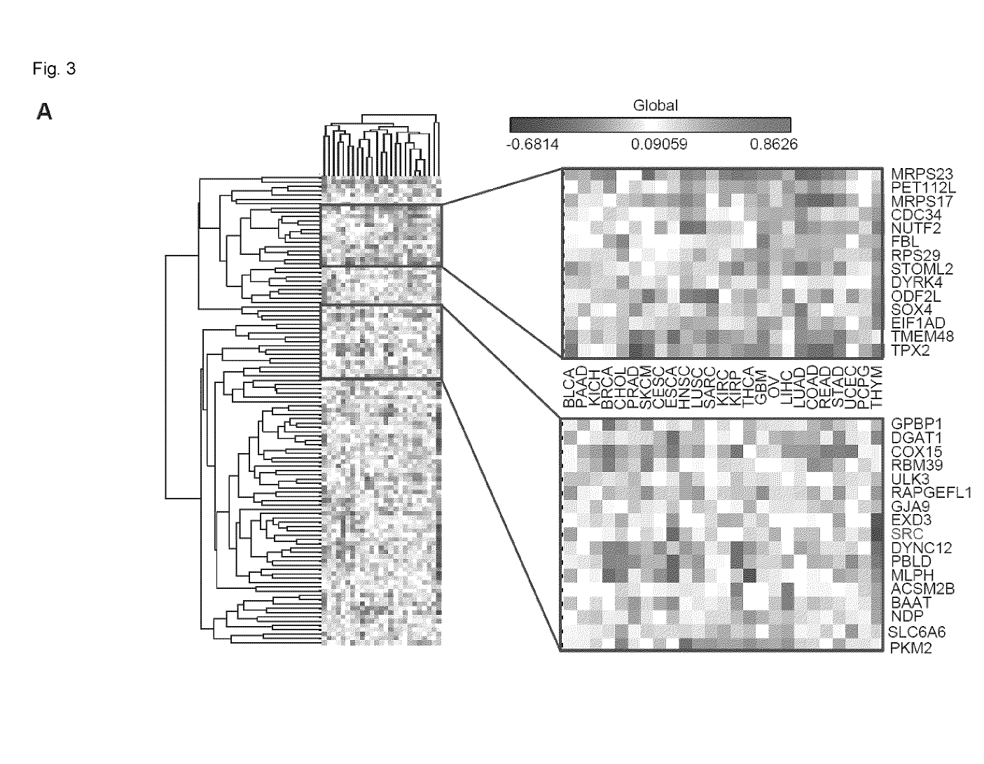

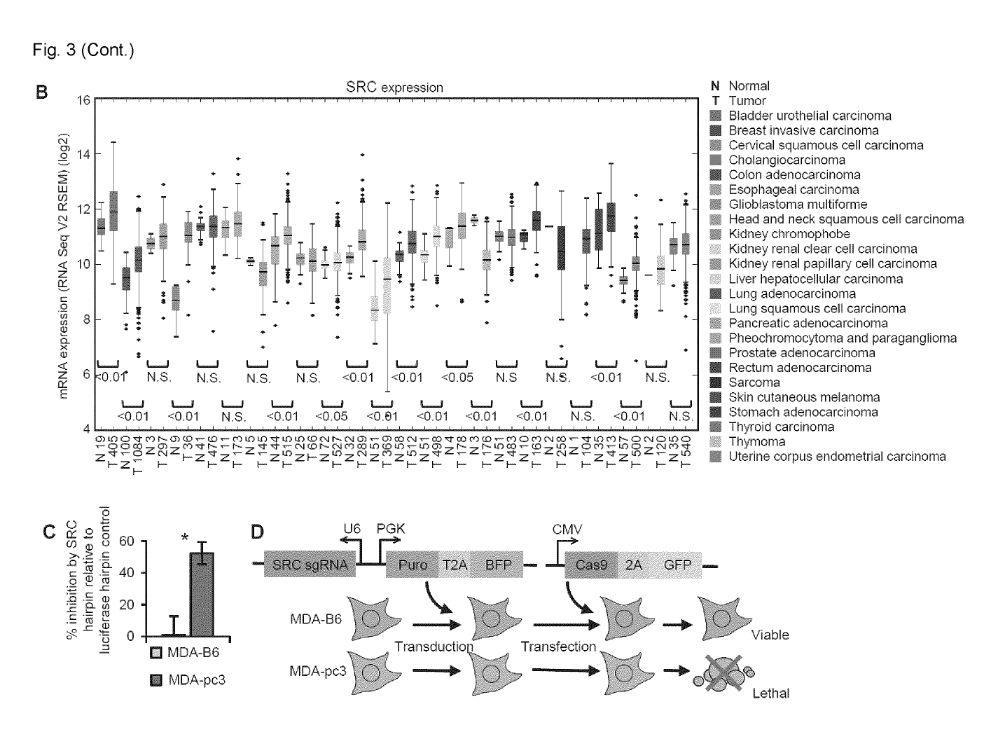

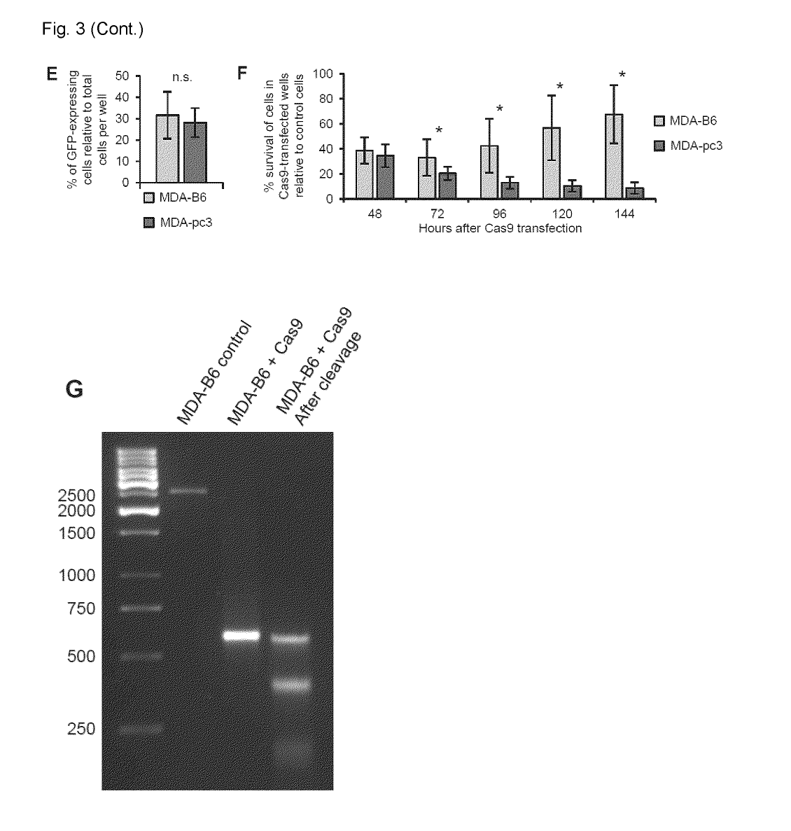

Application of tumor genome sequencing has identified loss-of-function alterations in cancer cells. While these alterations are difficult to target using direct interventions, they may be attacked with the help of the synthetic lethality (SL) approach. In this approach, inhibition of one gene causes lethality only when another gene is also completely or partially inactivated. The EPHB6 receptor tyrosine kinase has been shown to have anti-malignant properties and to be downregulated in multiple cancers, which makes it an attractive target for SL applications. As described in the Examples, a genome-wide SL screen combined with expression and interaction network analyses, identified genes in Table 1, a subset of which are shown in FIG. 3A, and include DDR2, SRC, ROCK2 and MET as SL partners of EPHB6 in triple-negative breast cancer (TNBC) cells. The experiments also reveal that this SL interaction can be targeted for example by small molecule SRC inhibitors, such as SU6656 and KX2-391, as well as MET inhibitors, such as ARQ197, and can be used to improve elimination of human TNBC tumors in a xenograft model. TNBC is an aggressive heterogeneous malignancy with a very high rate of patient mortality due to the lack of targeted therapies. Further, EPHB6 is downregulated in multiple malignancies suggesting that EPHB6 deficiency may be targeted by small molecule inhibitors in multiple cancers.

### DEFINITIONS

As used herein “EPHB6”, also referred to as “EPHB6 receptor” means the Ephrin type-B receptor 6, and includes all naturally occurring forms (e.g. isoforms) from all species, and particularly human including, for example, human EPHB6 which is encoded by the EPHB6 gene and for example has “Primary (citable) accession number” F8WCM8, the sequence of which is herein incorporated by reference. As used herein, EPHB6 may refer to the protein (also referred to as polypeptide), and/or the EPHB6 transcript as would be understood according to the context. For example, in methods measuring polypeptide levels, it would be understood that reference to EPHB6 or EPHB6 receptor is referring to EPHB6 polypeptide levels.

As used herein an “inhibitor” means any compound that is capable of inhibiting the expression and/or particularly an activity of a Table 1 molecule, preferably a polypeptide encoded by such gene, listed in Table 1. For example, a compound is an inhibitor if it reduces expression and/or activity by at least 50% compared to a control, for example a sample not treated with the inhibitor and includes for example inhibitors with an IC50 value at least in μM range.

As used herein “polypeptide listed Table 1” refers a polypeptide encoded by the corresponding gene associated with the Gene ID in Table 1 including all variants thereof.

As used herein, “Table 1 molecule” refers to a polypeptide or transcript encoded by the corresponding gene associated with the Gene ID in Table 1 including all variants thereof.

As used herein “kinase inhibitor” means any compound that is capable of inhibiting the expression and/or particularly the activity of a kinase for example by at least 50% compared to a control. Such inhibitor may, for example, interfere with gene transcription, processing (e.g. splicing, export from the nucleus and the like) and/or translation or may completely or partially inhibit kinase activity, particularly compounds that show a high potency (for example with an IC50 value at least in μM range). For example compounds that inhibit DDR2 (Discoidin domain receptor 2) kinase for example DDR2 kinase activity, are “DDR2 kinase inhibitors” and compounds that inhibit SRC kinase for example SRC kinase activity are “SRC kinase inhibitors”.

As used herein “SRC kinase” means a product of the human SRC gene.

“SRC” includes all naturally occurring forms (e.g. isoforms) from all species, and particularly human, including human SRC kinase encoded by the SRC gene in humans and for example having “Primary (citable) accession number” P12931, the sequence of which is herein incorporated by reference.

As used herein “SRC kinase inhibitor” means any compound that is capable of inhibiting the expression and/or activity of the SRC kinase for example by at least 50% compared to a control or a non SRC family member kinase. Such inhibitor may, for example, interfere with gene transcription, processing (e.g. splicing, export from the nucleus and the like) and/or translation or may completely or partially inhibit kinase activity, particularly compounds that show a high potency (e.g. low IC50 value). Examples include dasatinib, bosutinib (SKI-606), saracatinib (AZD530), SU6656, KX2-391 and/or posatinib (AP24534), PPI, PP2, Quercetin and/or pharmaceutically acceptable salts, solvates, and/or hydrates thereof. The SRC kinase inhibitor shows a high potency in SRC inhibition (for example with an IC50 value at least in μM range).

The term “KX2-391” as used herein means a compound having the formula:

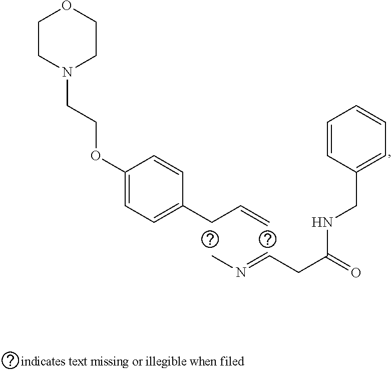

optionally including any salt thereof.

The term “SU6656” as used herein means a compound having the formula:

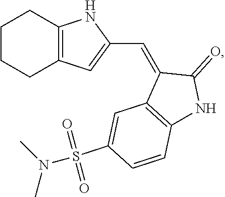 [SMILES: CN(C)S(=O)(=O)c1ccc2c(c1)/C(=C\c1cc3c([nH]1)CCCC3)C(=O)N2]

optionally including any salt thereof.

The term “PPI” as used herein means a compound having the formula:

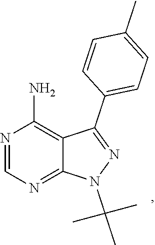 [SMILES: Cc1ccc(-c2nn(C(C)(C)C)c3ncnc(N)c23)cc1]

optionally including any salt thereof.

As used herein, “MET kinase”, also known as “c-MET”, “MET” or “hepatocyte growth factor receptor” includes all naturally occurring forms (e.g. isoforms) and splice versions from all species and particularly human including human MET kinase which is encoded by the MET gene in humans and has for example “Primary (citable) accession number” P08581, the sequence of which is herein incorporated by reference.

As used herein “MET kinase inhibitor” means any compound that is capable of inhibiting the expression and/or activity of MET kinase for example by at least 50% compared to a control. Such inhibitor may, for example, interfere with gene transcription, processing (e.g. splicing, export from the nucleus and the like) and/or translation or may completely or partially inhibit kinase activity, particularly compounds that show a high potency (e.g. low IC50 value). Examples include tivantinib (ARQ197), K252a, SU11274, AM7, PHA-665752, PF-2341066, foretinib, SGX523, MP470, crizotinib, cabozantinib, and/or pharmaceutically acceptable salts, solvates, and/or hydrates thereof. Also included are c-Met kinase inhibitors described in U.S. Pat. No. 9,238,571, incorporated herein by reference. The MET kinase inhibitor shows a high potency in MET inhibition (for example with an IC50 value at least in μM range).

The term “ARQ197” or “tivantinib” as used herein means a compound having the formula:

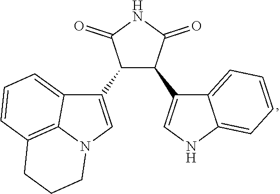 [SMILES: O=C1NC(=O)[C@@H](c2cn3c4c(cccc24)CCC3)[C@@H]1c1c[nH]c2ccccc12]

optionally including any salt thereof.

As used herein, “DDR2”, also known as “discoidin domain-containing receptor 2”, “DDR2 receptor” or “CD167b” includes all naturally occurring forms (e.g. isoforms) from all species and particularly human including human DDR2 kinase which is encoded by the DDR2 gene in humans and has for example “Primary (citable) accession number” A0A024R906, the sequence of which is herein incorporated by reference.

As used herein, the term “DDR2 kinase inhibitor” means any compound that is capable of inhibiting the expression and/or activity of a DDR2 kinase for example by at least 50% compared to a control. Such inhibitor may, for example, interfere with gene transcription, processing (e.g. splicing, export from the nucleus and the like) and/or translation or may completely or partially inhibit kinase activity, particularly compounds that show a high potency (e.g. low IC50 value). Examples include dasatinib and PB1 and/or pharmaceutically acceptable salts, solvates, and/or hydrates thereof. The DDR2 inhibitor shows a high potency in DDR2 inhibition (for example with an IC50 value at least in μM range).

As used herein, “ROCK2”, also known as “Rho associated coiled-coil containing protein kinase 2” includes all naturally occurring forms, and particularly human including human ROCK2 kinase which is encoded by the ROCK2 gene in humans and has for example “Primary (citable) accession number” O75116, the sequence of which is herein incorporated by reference.

As used herein, the term “ROCK2 kinase inhibitor” means any compound that is capable of inhibiting the expression and/or activity of a ROCK2 kinase for example by at least 50% compared to a control. Such inhibitor may, for example, interfere with gene transcription, processing (e.g. splicing, export from the nucleus and the like) and/or translation or may completely or partially inhibit kinase activity, particularly compounds that show a high potency (e.g. low IC50 value). Examples include Y27632 and fasudil and/or pharmaceutically acceptable salts, solvates, and/or hydrates thereof. The ROCK2 inhibitor shows a high potency in ROCK2 inhibition (for example with an IC50 value at least in μM range).

As used herein the phrase “deficiency in EPHB6 receptor levels” and the like means a decreased level of EPHB6 receptor protein and/or mRNA levels in a tumor tissue or cell sample optionally relative to normal control, optionally tumor adjacent normal tissue and/or normal cells. Optionally the decreased level in tumor tissue and/or tumor cells is at least 20% decreased, at least 30% decreased, at least 40% decreased, at least 50% decreased, at least 60% decreased, at least 70% decreased, at least 80% decreased, at least 90% decreased or more relative to normal tissue and/or normal cells, optionally compared to a mean expression level in the matching normal tissue. The decreased level can also be undetectable using a standard assay or below a selected threshold. Deficiency can also be assessed by determining if the EPHB6 receptor promoter is methylated which can reduce and/or shut off transcription and thereby reduce levels. Accordingly in methods where promoter methylation is assessed or a selected threshold is used, comparison to a control is not strictly necessary but may be employed.

As used herein “a biological sample” means any sample from a subject such as a human and comprises cancer and/or tumor cells, including for example a tumor tissue sample such as a biopsy, tissue slice, cancer cell smear, circulatory tumor cells, surgical specimen, etc.

The term “antibody” as used herein is intended to include synthetic antibodies, monoclonal antibodies, polyclonal antibodies, human, humanized and chimeric antibodies. The antibody may be from recombinant sources and/or produced in transgenic animals. The antibody can be any species or a human antibody for example derived from display technologies such as phage antibody display libraries. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. Antibody fragments mean binding fragments.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art. For example, in the following passages, different aspects are defined in more detail. Each aspect so defined may be combined with any other aspect or aspects unless clearly indicated to the contrary. In particular, any feature indicated as being preferred or advantageous may be combined with any other feature or features indicated as being preferred or advantageous. For example, reference to an inhibitor of a Table 1 molecule can be combined with any other inhibitor of a Table 1 molecule in any embodiment described herein. For example, any method of detecting EPHB6 receptor level can be combined with any inhibitor of a Table 1 molecule. For example, any of the inhibitors listed herein, any combination of inhibitors, any cancer and any subgroup of cancers listed herein can be combined.

### METHODS AND PRODUCTS

As disclosed herein, the present disclosure provides methods for identifying patients likely to respond an inhibitor of a Table 1 molecule, such as a SRC kinase inhibitor or a MET kinase inhibitor. The methods described involve assessment and/or measurement of EPHB6 levels in a biological sample comprising tumor and/or cancer cells.

An aspect includes a method of identifying a subject with a cancer eligible for treatment with an inhibitor of a Table 1 molecule, comprising testing a biological sample from the subject for a deficiency in EPHB6 receptor levels, wherein the subject is eligible for treatment with an inhibitor of a Table 1 molecule if EPHB6 receptor levels in the biological sample are deficient.

An aspect includes a method of identifying a subject with a cancer eligible for treatment with a SRC kinase inhibitor comprising testing a biological sample from the subject for a deficiency in EPHB6 receptor levels, wherein the subject is eligible for treatment with SRC kinase inhibitor if EPHB6 receptor levels in the biological sample are deficient.

Another aspect includes a method of identifying a subject with a cancer eligible for treatment with a MET kinase inhibitor comprising testing a biological sample from the subject for a deficiency in EPHB6 receptor levels, wherein the subject is eligible for treatment with MET kinase inhibitor if EPHB6 receptor levels in the biological sample are deficient.

In an embodiment, the method comprises monitoring the subject's tumor for EPHB6 receptor levels, wherein the subject is eligible for treatment with an inhibitor of a Table 1 molecule, for example a SRC kinase inhibitor or a MET kinase inhibitor, if the subsequent sample tested for EPHB6 receptor levels is deficient.

In an embodiment, the EPHB6 receptor level tested is EPHB6 receptor polypeptide. In another embodiment, the EPHB6 receptor level tested is EPHB6 receptor transcript.

In an embodiment, testing a biological sample from the subject for a deficiency in EPHB6 receptor levels comprises binding a specific binding agent such as an antibody to EPHB6 polypeptide (e.g. extracellular domain) or an agent binding to EPHB6 transcript in the biological sample, forming a complex between the specific binding agent and EPHB6 polypeptide or EPHB6 transcript and measuring the level of EPHB6 polypeptide or transcript complex in the biological sample. The measured EPHB6 polypeptide or transcript level is then used in the assessment of whether the subject is eligible for treatment with an inhibitor of a Table 1 molecule, wherein a deficiency or absence of EPHB6 polypeptide or EPHB6 transcript levels as compared to a normal control, optionally tumor adjacent normal tissue and/or normal cells, is indicative the subject may be eligible for treatment with an inhibitor of a Table 1 molecule.

Another aspect includes a method for personalizing a cancer treatment, comprising binding a specific binding agent to EPHB6 polypeptide or EPHB6 transcript in a biological sample, measuring the level of EPHB6 polypeptide or EPHB6 transcript in the biological sample; using the measured level of EPHB6 polypeptide or EPHB6 transcript to select a cancer treatment, wherein a deficiency or absence of EPHB6 polypeptide or EPHB6 transcript as compared to a normal control, optionally tumor adjacent normal tissue and/or normal cells, is indicative the subject may be eligible for treatment with an inhibitor of a Table 1 molecule, and providing a personalized cancer treatment.

In an embodiment, the inhibitor is an inhibitor of DDR2, ROCK2, SRC and/or MET.

In an embodiment, the inhibitor is a SRC kinase inhibitor. In an embodiment, the inhibitor is a MET kinase inhibitor. In an embodiment, the inhibitor is a DDR2 kinase inhibitor. In an embodiment, the inhibitor is a ROCK2 kinase inhibitor.

Several molecules targeting MET have been evaluated in early phase clinical trials including small compound kinase inhibitors, biological antagonists and monoclonal antibodies targeting either the ligand or the receptor. An example is ARQ197.

Accordingly in an embodiment, the inhibitor is a small molecule inhibitor inhibitor or an antibody that inhibits a molecule in Table 1.

In an embodiment, the inhibitor is an antibody such as a monoclonal antibody that inhibits MET kinase. In an embodiment, the inhibitor is an antibody such as a monoclonal antibody that inhibits DDR2 kinase.

Another aspect includes a method of treating a cancer in a subject comprising: administering an effective amount an inhibitor of a Table 1 molecule to a subject in need of such a treatment having a cancer with decreased expression of EPHB6. The subject in need of such treatment is identified by evaluating the level of EPHB6 receptor in a biological sample of a subject suspected of having cancer, having cancer or being prone to having cancer.

A further aspect includes a method of treating a cancer in a subject comprising: administering an effective amount of a SRC kinase inhibitor to a subject in need of such a treatment, wherein the subject in need of such treatment is identified by evaluating the level of EPHB6 receptor in a biological sample of a subject suspected of having cancer, having cancer or being prone to having cancer, and wherein a deficiency in EPHB6 receptor levels in the biological sample optionally compared to a control indicates responsiveness of the subject to the SRC kinase inhibitor.

A further aspect includes a method of treating a cancer in a subject comprising: administering an effective amount of a MET kinase inhibitor to a subject in need of such a treatment, wherein the subject in need of such treatment is identified by evaluating the level of EPHB6 receptor in a biological sample of a subject suspected of having cancer, having cancer or being prone to having cancer, and wherein a deficiency in EPHB6 receptor levels in the biological sample optionally compared to a control indicates responsiveness of the subject to the MET kinase inhibitor.

In an embodiment, the biological sample comprises or is a tumor sample. In an embodiment, the biological sample is a biopsy such as a fine needle aspirate or an image guided biopsy. In an embodiment, the biological sample is a tissue slice, a cancer cell smear or a surgical specimen. In an embodiment, the biological sample is frozen sample, a fresh sample or a fixed sample.

In an embodiment, the subject administered an effective amount of an inhibitor of a Table 1 molecule optionally a SRC kinase inhibitor or a MET kinase inhibitor is a subject with a cancer having a deficiency of EPHB6 polypeptide and/or EPHB6 transcript levels.

A further aspect includes a method of treating a cancer in a patient, comprising testing for a deficiency in EPHB6 receptor levels in a biological sample from the patient and administering a therapeutically effective amount of an inhibitor of a Table 1 molecule optionally a SRC kinase inhibitor or a MET kinase inhibitor to the patient if the sample tests positive for a deficiency EPHB6 polypeptide and/or EPHB6 transcript levels.

In an embodiment, the deficiency in EPHB6 receptor levels is determined by measuring the level of EPHB6 receptor protein or mRNA (for example by making cDNA) in tumor tissue and/or cancer cells.

In an embodiment, a subject is deficient in EPHB6 if the level is at least 20% decreased, at least 30% decreased, at least 40% decreased, at least 50% decreased, at least 60% decreased, at least 70% decreased, at least 80% decreased, at least 90% decreased or more relative to normal tissue and/or normal cells. For example, the EPHB6 receptor level is at least 20% decreased, at least 30% decreased, at least 40% decreased, at least 50% decreased, at least 60% decreased, at least 70% decreased, at least 80% decreased, at least 90% decreased or more compared to EPHB6 mean expression level in matching normal tissue. The decreased level can also be undetectable using a standard assay or below a selected threshold.

EPHB6 receptor is a cell surface receptor and polypeptide levels can be measured for example by immunohistochemistry, flow cytometry, western blot and other antibody or ligand based methods for example including the methods described in the Examples. The EPHB6 receptor level detected in the biological sample refers the EPHB6 receptor level associated with the cancer cells.

In an embodiment, the EPHB6 level is measured by immunohistochemistry.

EPHB6 receptor levels may be measured using any antibody based methods. For example, any anti-EPHB6 antibody that detects an epitope in the extracellular domain of EPHB6 can be used.

In an embodiment, the method comprises obtaining a biological sample, contacting the sample with an anti-EPHB6 antibody to form an anti-EPHB6 antibody: EPHB6 complex with any EPHB6 in the sample, and measuring the level of anti-EPHB6 antibody: EPHB6 complex.

In another embodiment, the method comprises obtaining a biological sample, optionally a tumor sample, with a primary antibody to form and anti-EPHB6 antibody: EPHB6 complex and further contacting the anti-EPHB6 antibody: EPHB6 complex with a secondary antibody to detect the EPHB6-antibody complex, and determining the sample as deficient in EPHB6 receptor levels if the presence of EPHB6-antibody complex is not detected.

In an embodiment, the antibody such as the primary and/or secondary antibody is labeled.

In an embodiment, the EPHB6 receptor level is determined by flow cytometry and comprises isolating cancer cells from the biological sample, optionally cancer cells in a tumor sample, incubating the cancer cells with a primary anti-EPHB6 antibody, optionally incubating the labeled cancer cells with a secondary antibody, optionally a FITC-conjugated antibody and conducting flow cytometry. For example, the level of fluorescence emitted by the labeled cancer cells can be compared against the level of fluorescence emitted by cancer cells.

In an embodiment, the inhibitor is to a cell surface receptor listed in Table 1.

Deficiency in EPHB6 receptor can also be measured by assessing promoter methylation. Promoter methylation reduces and/or prevents transcription and detecting promoter methylation of the EPHB6 receptor promoter indicates a deficiency in EPHB6 receptor levels. Methods for measuring promoter methylation are known and include for example mass spectrometry, methylation specific PCR(MSP) bishulphite conversion based assays, ChIP-on chip assays, methylated DNA immunoprecipitation as well as methods using solid state nanopores.

In an embodiment, EPHB6 receptor levels are detected using a combination of methods described herein.

In an embodiment, the patient was previously tested and determined as having a cancer deficient in EPHB6 levels.

In an embodiment, the method further comprises retesting at a later time point the EPHB6 receptor levels in a biological sample of the patient and treating patient with an inhibitor of Table 1 molecule if a deficiency in EPHB6 receptor levels in the biological sample is detected, or if a decrease in EPHB6 receptor levels is detected in the biological sample compared to EPHB6 receptor levels measured in a biological sample obtained at an earlier time point. In an embodiment, the biological sample obtained at a later time point is from a metastatic tumor.

Yet a further aspect is a method of personalizing treatment in a subject having or suspected of having cancer comprising measuring EPHB6 receptor levels in a biological sample obtained from the subject, optionally comparing the measured EPHB6 to a control, treating the subject with an inhibitor of a Table 1 molecule optionally a SRC kinase inhibitor or a MET kinase inhibitor when the level of EPHB6 is deficient, and otherwise treating the subject with an alternate treatment, for example when the level of EPHB6 receptor is comparable or increased compared to a control such as adjacent normal tissue.

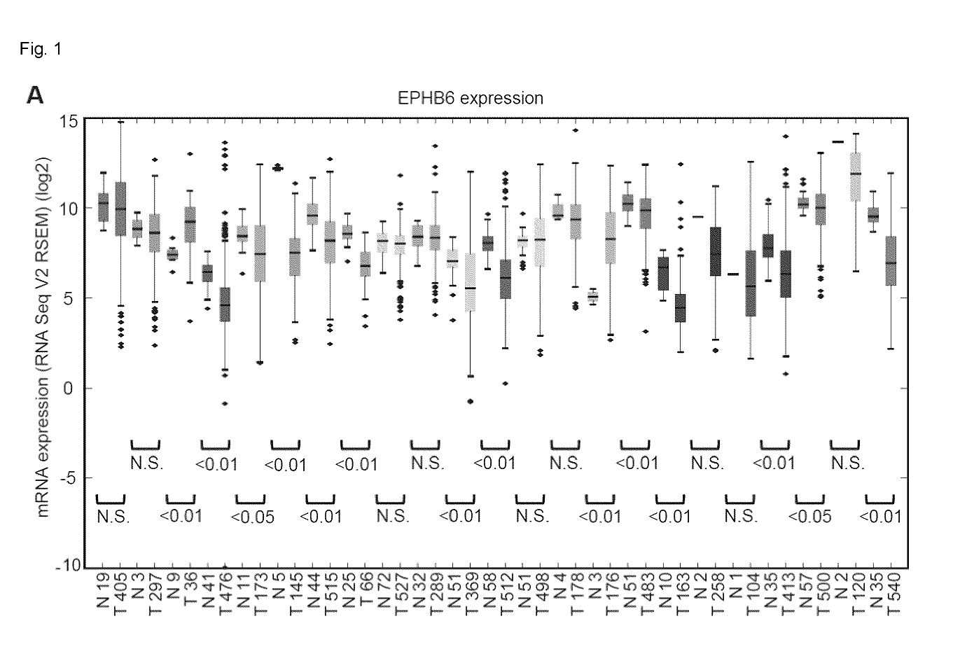

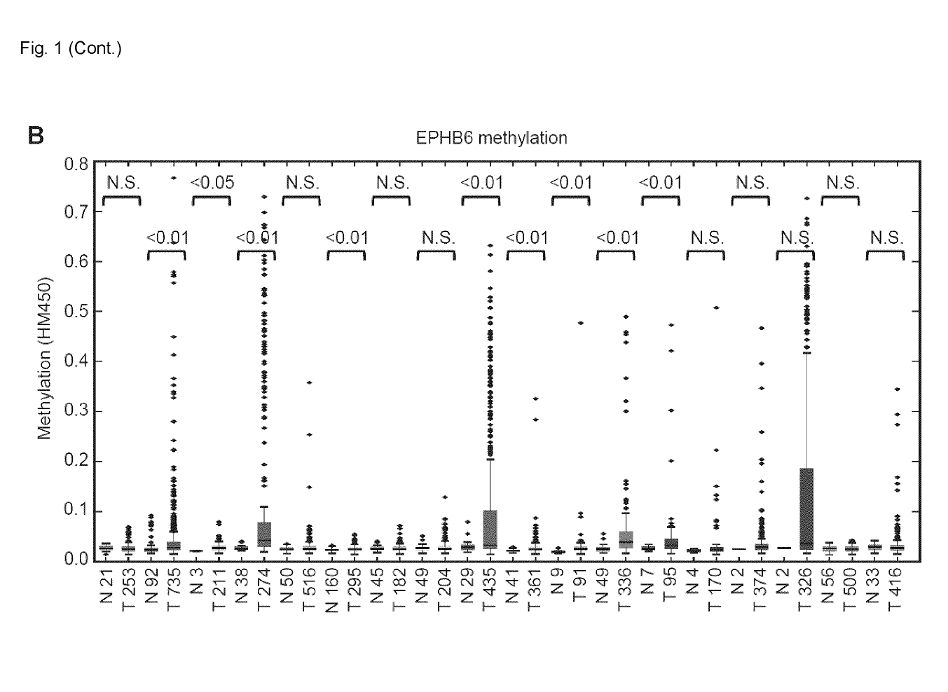

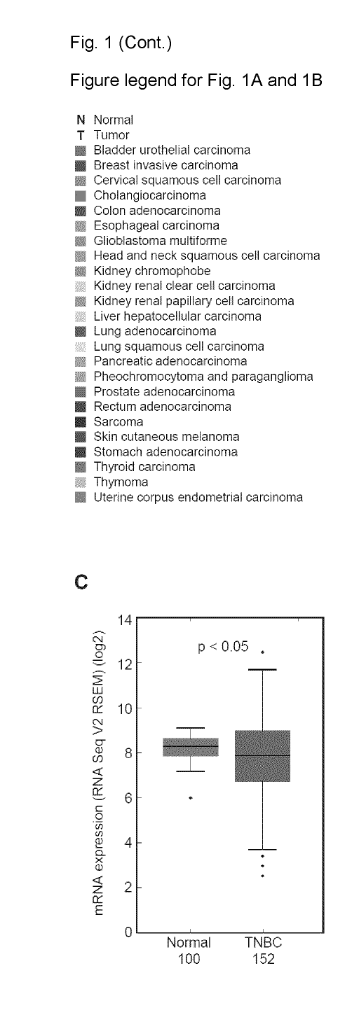

In an embodiment, the cancer is selected from breast cancer, including for example invasive breast cancer and/or triple negative breast cancer (TNBC); lung cancer such as metastatic lung cancer, melanoma, prostate cancer, ovarian carcinoma, gastric cancer, colon and neuroblastoma including aggressive neuroblastoma. In an embodiment the cancer is selected from a cancer with decreased EPHB6 expression listed in FIG. 1a, for example colon adenocarcinoma, esophageal carcinoma, glioblastoma multiforme, head and neck squamous cell carcinoma, kidney chromophobe, liver hepatocellular carcinoma, lung adenocarcinoma, prostate adenocarcinoma, rectum adenocarcinoma, stomach adenocarcinoma, thyroid carcinoma and uterine corpus endometrial carcinoma. In an embodiment, the cancer is selected from a cancer having increased EPHB6 methylation listed in FIG. 1b, for example breast invasive carcinoma, cervical squamous cell carcinoma, colon adenocarcinoma, kidney renal clear cell carcinoma, lung adenocarcinoma, lung squamous cell carcinoma, pancreatic adenocarcinoma, prostate adenocarcinoma and rectum adenocarcinoma.

In some embodiments, for example where EPHB6 receptor levels are known to be decreased in greater than 25%, greater than 30%, greater than 35%, greater than 40%, greater than 45% or greater than 50% in patients with a particular cancer type, for example triple negative breast cancer, treatment may proceed without confirmed deficiency in EPHB6 polypeptide or transcript levels.

In an embodiment, the SRC kinase inhibitor is selected from dasatinib, bosutinib (SKI-606), saracatinib (AZD530), SU6656, KX2-391 and/or posatinib (AP24534), PPI, PP2, Quercetin and/or pharmaceutically acceptable salts, solvates, and/or hydrates thereof.

In an embodiment, the SRC kinase inhibitor is SU6656 or KX2-391.

As described in more detail below, use of inhibitors for example of the SRC kinase, in accordance with the present invention is not limited to the herein described or further known inhibitors. Accordingly, also yet unknown inhibitors may be used in accordance with the present invention. Such inhibitors may be identified by the methods described and provided herein and methods known in the art, like high-throughput screening using biochemical assays for inhibition of the SRC kinase.

In an embodiment, the MET kinase inhibitor is selected from tivantinib (ARQ197), K252a, SU11274, AM7, PHA-665752, PF-2341066, foretinib, SGX523, MP470, crizotinib, cabozantinib, and/or pharmaceutically acceptable salts, solvates, and/or hydrates thereof.

In an embodiment, the MET kinase inhibitor is ARQ197.

As described in more detail below, use of inhibitors for example of the MET kinase, in accordance with the present invention is not limited to the herein described or further known inhibitors. Accordingly, also yet unknown inhibitors may be used in accordance with the present invention. Such inhibitors may be identified by the methods described and provided herein and methods known in the art, like high-throughput screening using biochemical assays for inhibition of the MET kinase.

As described in the Examples, other molecules including DDR2 and ROCK2 were identified. Accordingly another aspect includes using the methods described herein replacing or further assessing the level of one or more of DDR2 and ROCK2. Statistically significant targets that demonstrated SL with EPHB6 receptor deficient cells are shown in Table 1.

Accordingly another aspect includes using the methods described herein and further assessing the level of one or more of the molecules in Table 1. In an embodiment, the methods described herein are used further assessing the level of one or more molecules in FIG. 3A.

The treatment methods can also be combined with other treatments including surgery, radiation, chemotherapy and the like. In an embodiment, an inhibitor of a Table 1 molecule optionally a SRC kinase inhibitor or a MET kinase inhibitor is administered in a combination therapy. In an embodiment the combination therapy comprises chemotherapy. For example, the treatment can be combined with any known treatment for the particular cancer. In an embodiment, the combination therapy comprises administering two or more inhibitors herein described.

In an embodiment, the subject is a mammal. In an embodiment the subject is a human.

Also provided are screening methods for identifying putative inhibitors, for example inhibitors of a Table 1 molecule, optionally a SRC kinase inhibitor or MET kinase inhibitor using for example cells deficient and not deficient in EPHB6 levels.

In an embodiment, cells deficient and not deficient are cultured with a test compound and cell expansion and/or cell death is measured and the test compound that reduces cell expansion or induces cell death in EPHB6 receptor deficient cells is identified as a putative inhibitor.

In an embodiment, the screening assay comprises:
contacting a control cancer cell sample with a test candidate;
contacting a test cancer cell sample deficient in EPHB6 receptor levels with the test candidate;
measuring an effect of the test candidate on the control cancer cell sample and on the test cancer cell sample;
comparing the effect of the test candidate on the control cancer cell sample and on the test cancer cell sample; and
identifying the test candidate as a putative inhibitor, optionally a putative SRC kinase inhibitor or a putative MET kinase inhibitor, when the effect measured is greater on the test cancer cell sample compared to the control cancer cell sample.

In an embodiment, the screening assay is for selecting a candidate treatment for EPHB6 deficient cancer cells.

In an embodiment, the screening assay further comprises contacting a second control cancer cell sample and a second test cancer cell sample with a known inhibitor of a Table 1 molecule, optionally a known SRC kinase inhibitor or a known MET kinase inhibitor; measuring an effect of the test candidate on the second control cancer cell sample and on the second test cancer cell sample, identifying the test candidate as a putative inhibitor when the effect measured is at least comparable to the known inhibitor.

In an embodiment, the effect measured is cell death and/or decreased in cell proliferation and the test candidate that induces cell death and/or inhibits cell proliferation, optionally by at least a comparable level to the known inhibitor, is identified as a putative inhibitor.

Inducing cell death or inhibiting cell proliferation can be measured by a variety of assays, including assays described herein as well as apoptotic assays and necrotic assays, measured for example using fluorescent dyes, flow cytometry, assessing nuclear morphology, etc.

In an embodiment, the control cancer cell sample is adjacent normal tissue or a non EPHB6 deficient cancer cell sample and the test cancer cell sample is a test tumor, the effect measured is tumor volume, and the test candidate that decreases the tumor volume and/or suppresses tumor growth, optionally by at least a comparable level to the known inhibitor, is identified as a putative inhibitor.

Although process steps, method steps, algorithms or the like may be described (in the disclosure and/or in the claims) in a sequential order, such processes, methods and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

The methods and materials used in the other Examples are provided here.

### MATERIALS AND METHODS

### Antibodies and Reagents

Anti-phospho-SRC was from Life Technologies (Burlington, ON, Canada). Anti-c-SRC, anti-β-tubulin and SU6656 were from Santa Cruz Biotechnology (Dallas, Tex., USA). Human anti-EPHB6 antibody, matching sheep IgG control, FITC-conjugated anti-sheep secondary antibody, and resazurin were from R&D Systems (Minneapolis, Minn., USA). BSA was from BioShop Canada Inc. (Burlington, ON, Canada). KX2-391 was from Selleckchem (Houston, Tex., USA). 7-AAD kit was from BD Biosciences (Mississauga, ON, Canada). Dimethyl sulfoxide (DMSO) and polybrene were from Sigma-Aldrich (St. Louis, Mo., USA). Propidium iodide and puromycin were from ThermoFisher Scientific (Burlington, ON, Canada). Pooled screen shRNAs and constructs were derived from the RNAi Consortium lentiviral library (Sigma-Aldrich). sgRNA constructs encoding BFP were from MilliporeSigma/welcome trust Sanger (Sigma-Aldrich). pLD-GFP-puro and pLD-RFP-puro expression constructs were previously described [3]. The GeneArt Genomic cleavage detection kit was from ThermoFisher Scientific.

### Cell Lines and Culture Conditions

MDA-MB-231 and BT-20 cells were purchased from the American Type Culture Collection (Manassas, Va., USA). Cells were passaged for less than three months at a time following resuscitations and therefore, no additional authentication was performed. Both MDA-MB-231 and BT-20 monolayer cultures were maintained in the DMEM medium containing 10% FBS (Gibco, Life Technologies), 1% penicillin/streptomycin (Gibco, Life Technologies) and 1 mM sodium pyruvate (HyClone, GE Life Sciences,).

### Stable Cell Lines and Lentiviral Transduction

Stable MDA-MB-231 cell lines with restored EPHB6 expression were generated by transfecting MDA-MB-231 cells with the pcDNA3 expression vector encoding wild-type EPHB6 (MDA-B6) or Myc-tagged EPHB6 (MDA-B6-M). Transfection with the empty vector was used as a control (MDA-pc3). Stable EPHB6 knockdowns were generated using EPHB6-targeting shRNA encoded in lentiviral particles (Santa Cruz Biotechnology, Dallas, Tex., USA). Cells were transduced using 10 μg/mL polybrene (Sigma-Aldrich), followed by 5 days of selection with 10 μg/mL puromycin (Sigma-Aldrich). Transduction with SRC-targeting shRNA constructs and with GFP- or RFP-encoding cDNAs, required preparation of lentiviral particles. Lentiviral particles were generated by transfection of HEK-293T cells, grown in 10 cm plates to ˜70% confluence with psPAX2, pMD2.G, and with the lentiviral vector encoding the genes of interest. Transfection took place in 10 mL of tissue culture medium with 1,400 μL Opti-Mem (Gibco, Life Technologies) and 93.6 μl X-treamGENE 9 DNA Transfection Reagent (Roche, Mississauga, ON, Canada). Medium was changed 18 hours later and replaced with DMEM containing 2% (w/v) bovine serum albumin (BSA) and viral particles were collected 48 h and 72 h after transfection. MDA-B6 and MDA-pc3 cells were transduced with the lentiviral particles by incubation overnight in medium containing 8 μg/mL polybrene. The transduction medium was removed and transduced cells were incubated for 48 h in cell culture medium containing 2 μg/mL puromycin.

### CRISPR/Cas9 Analysis

MDA-B6 and MDA-pc3 cells were seeded in 6-well plates and transduced with src-targeting sgRNAs lentiviral constructs that also encoded BFP in the presence of 8 μg/mL of polybrene. Following 48 h of selection with 2 μg/mL puromycin, selected cells were seeded in 96-well optical bottom plates (ThermoFisher Scientific), allowed to adhere for 24 h, and transfected with CMV-Cas9-2A-GFP (Sigma CAS9GFPP-1EA) using the Lipofectamine LTX and Plus Reagent kit (ThermoFisher Scientific) according to the manufacturer's instructions. Cells were imaged every 24 hours for six days after transfection using the ImageXpress Micro XLS widefield automated fluorescence microscope (Molecular Devices, Sunnyvale, Calif., USA) to capture BFP and GFP signals. Cell expressing BFP or co-expressing BFP and Cas9-GFP were quantified using MetaXpress version 6 (Molecular Devices). src knockout was confirmed using the GeneArt Genomic cleavage detection kit (ThermoFisher Scientific) following the manufacturer's instructions.

### Drug Sensitivity Assays

MDA-MB-231 and BT-20 cell monolayers were incubated in 96-well plates for 72 h and 96 h, respectively, with indicated concentrations of KX2-391 or SU6656, or matching volumes of DMSO as a solvent control. Treated cells were stained using resazurin by following the manufacturer's instructions and fluorescence was measured using a SpectraMax M5 microplate reader.

### Western Blotting

Cells were rinsed with ice-cold PBS and lysed using lysis buffer containing 0.1 M EDTA, 0.3 M Tris, 0.1 M NaCl, 6 mM PMSF, and 3 mM sodium ortho-vanadate. Cell debris were removed by centrifugation. For immunoprecipitation, 2-3 μg of required antibody, with 25 μL of protein G Sepharose beads (GE Healthcare Life Sciences, Baie d'Urfe, QC, Canada) were added. Samples were rotated at 4° C. overnight and beads were washed three times with lysis buffer. Cell lysates or immunoprecipitates were resolved using SDS-PAGE, followed by transfer to nitrocellulose membranes (Amersham, GE Healthcare Life Sciences). Membranes were blocked with 5% non-fat dry milk in 0.1% PBS/Tween-20, or with 5% BSA in TBS/Tween-20 and incubated overnight with primary antibodies at 4° C. At this stage, membranes were rinsed 3 times with PBS or TBS, incubated for 1 h with fluorescently labeled secondary antibodies (LI-COR Biotechnology, Guelph, ON, Canada) and protein images were acquired using the LI-COR Odyssey imaging system (LI-COR Biotechnology). Figures were generated using the Odyssey, Carestream and PowerPoint software. Cropping of Western blot images was done with PowerPoint. Brightness and contrast were adjusted in western blot images using Carestream and Powerpoint software to optimize image presentation. Western blotting with anti-tubulin was used as a loading control.

### Drug Sensitivity Assays with Fluorescent Cells

For color assays, MDA-B6-GFP and MDA-pc3-RFP, or MDA-B6-RFP and MDA-pc3-GFP cells were co-seeded in equal numbers in 12-well plates at indicated cell densities. Seeded cells were incubated for 72 h with 25 nM KX2-391 and a matching volume of DMSO. Treated cells were collected and quantitated by flow cytometry. Results were analyzed using the FlowJo software (FLOWJO LLC, Ashland, Oreg., USA).

### Monitoring Expression of EPHB6 on the Cell Surface

To confirm cell surface expression of EPHB6 in MDA-B6 and MDA-B6-M, cells were collected with 2 mM EDTA, washed with serum-free media, and incubated with anti-EPHB6 or matching IgG control for 40 minutes on ice. Labeled cells were washed twice with serum-free media, and incubated with FITC-conjugated secondary antibody for 30 minutes on ice in the dark. Cells were then washed twice with serum-free media and suspended in PBS for analysis by flow cytometry. Results were analyzed using the FlowJo software (FLOWJO LLC).

### Cell Death Assays

For propidium iodide (PI) staining, MDA-MB-231 and BT-20 cells were incubated in glass-bottom plates (MatTek, Ashland, Mass., USA) for 72 h and 96 h, respectively, with KX2-391 and matching volumes of DMSO. Cells were then incubated with 2.7 μg/ml PI for 12 minutes and washed with phenol red-free medium. The amount of PI-stained cells was analyzed by microscopy using a Zeiss Observer Z1 at 200× magnification. Brightness of presented confocal microscopy images was adjusted using the Zen 2012 Software (version 8.0) to optimize the visualization of PI staining. PI-stained cells were counted in at least 10 randomly captured frames, normalized on the total number of cells in matching frames and compared between DMSO controls and treated cells.

For 7-AAD staining, MDA-MB-231 and BT-20 cells were incubated in 6-well plates for 72 h and 96 h, respectively, with 25 nM KX2-391 and matching volumes of DMSO. Cells were collected and stained with 7-AAD according to the manufacturer's instructions, prior to flow cytometry analysis. Results were analyzed using the FlowJo software (FLOWJO LLC).

### Tumor Xenograft Studies and Immunohistochemistry

Breeder pairs of NOD SCID gamma mice were purchased from The Jackson Laboratory and a colony was established at the Laboratory Animal Services Unit, University of Saskatchewan. Mice were housed in sterile cages and maintained in pathogen-free aseptic rooms, while being fed autoclaved food pellets and water ad libitum. All animal protocols were reviewed and approved by the University of Saskatchewan Animal Research Ethics Board. Xenograft tumors were established by injection of 1×10⁶ MDA-B6 or MDA-pc3 in 100 μL PBS into the mammary fat pads of 4-6 week old female animals. Treatments with KX2-391 were initiated when tumors became palpable. Mice were fed with either KX2-391 (5 mg/kg) in DMSO/water solvent or a matching volume of the solvent. Treatments were administered orally twice a day. Digital caliper measurements were taken every 3 days and tumor volume was calculated by the formula A/2*B² (where A and B were the long and short diameters of the tumor respectively). At the end of the experiments animals were sacrificed and tumors were removed. Tumors were fixed in 10% buffered formalin for paraffin embedding.

For the immunohistochemical staining, tumors were dissected and fixed in 10% neutral- buffered formalin for 24-48 h. The tumors were paraffin embedded, sectioned to 4 μm thickness, and affixed on the slide. Simultaneous dewaxing and antigen retrieval was performed on the Dako PT Link using Target Retrieval Solution-High pH (Dako Canada, Burlington, ON, Canada). Staining was performed on the Dako Autostainer Link using anti-CD34 (Abcam, Toronto, ON, Canada) antibody and the Dako FLEX DAB+Detection Kit. In each stained tumor section, 12, 3, 6 and 9 o'clock fields were imaged at 100× magnification and the density of stained blood vessels per field was quantified using the Image-Pro Premier software.

### Expression Analysis

Expression data from TCGA datasets for different cancer types was collected in regard to both EPHB6 and SRC. The distribution was plotted for both tumor patients and normal patients. TCGA methylome data was also collected for EPHB6 and the distribution was plotted for both tumor patients and normal patients. Ovarian cancer that was analyzed in the correlation clustergram (FIG. 3A) is not included in the expression analysis due to the unavailability of data in matching normal tissue.

### Pooled Screening

Pooled shRNA screening was done as previously described [3]. Briefly, MDA-B6, MDA-B6-M, and MDA-pc3 cells were transduced with lentiviral particles containing a 90 K shRNA library with 200× hairpin representation. Cells were passaged for 17 days and genomic DNA was collected at T0, T10, and T17 for analysis. Genomic DNA was amplified by large-scale PCR. The amplification PCR reaction was carried out by denaturing once at 98° C. for 3 minutes, followed by (98° C. for 10 seconds, 55° C. for 15 seconds, 72° C. for 15 seconds) x29, 72° C. for 5 minutes, then cooling to 4° C. Amplification products were purified and digested with Xhol (New England Biolabs, Whitby, ON, Canada). The stable half-hairpins were purified and probe hybridization was carried out on UT-GMAP 1.0 microarrays (Affymetrix Inc, Santa Clara, Calif., USA).

### Computational Scoring of Pooled Screens

For each hairpin, the signal intensity was normalized and converted to log2 scale for each time point of both MDA-wild type, and MDA-pc3 samples. Note that the MDA-wild type samples were either MDA-B6 or MDA-B6-M. Hairpins whose signal was below the background (i.e. log2 scale of less than 8) at time point TO were discarded. Likewise, hairpins with fold-change greater than or equal to 1.25 at a time point relative to the corresponding previous time point were also discarded. For each replicate, the difference of cumulative change (DCC) between the MDA-pc3 and MDA-wild type conditions were calculated for time points relative to the corresponding previous time point using the formula:

$$DCC = {\sum }_{t = 1}^{T} ( {x}_{t , k}^{pc 3} - {x}_{t - 1 , k}^{pc 3} ) - {\sum }_{t = 1}^{T} ( {x}_{t , k}^{w} - {x}_{t - 1 , k}^{w} )$$

where x_{t,k}^{pc3} is the normalized signal intensity at time point t ∈(0, . . , T) and for replicate k ∈(1, . . , K) for MDA-pc3 samples. Likewise, x_{t,k}^{w} represent the same for MDA-B6 or MDA-B6-M samples. The DCC fitness score was then calculated for each gene by taking the two hairpin DCC values that were the most negative values for that gene. DCC_{g}=arg min_{h,h′}[DCC_{g,h}+DCC_{g,h′}]/2

Next, the permutation test was performed by randomly shuffling the DCC scores. This process was repeated and an empirical distribution of the DCC fitness scores over all of the genes was constructed. Finally, significant p-values for each observed fitness score were estimated as the frequency of randomized, shuffled DCC with more negative scores.

$$p = \frac{1}{NL} {\sum }_{r = 1}^{NL} I ( {DCC}_{r} < {DCC}_{g} )$$

where N is the number of genes, L is the number of repeats done to construct an empirical distribution, DCCᵣ is the randomized shuffle with more negative score, and I() is a binary indicator that give 1 for a true statement, and 0 otherwise.

### Statistical Analysis

Student's t-test was performed for statistical analyses, until otherwise indicated. Data are presented as mean ±SD.

### Example 2

### Results

Genome-wide shRNA screen reveals synthetic lethal interactions of the EPHB6 receptor in TNBC cells

A systematic analysis of the gene expression data from The Cancer Genome Atlas (TCGA) dataset expanded previous observations and confirmed that EPHB6 is indeed downregulated in multiple tumor types (FIG. 1A) including for example colon adenocarcinoma, esophageal carcinoma, glioblastoma multiforme, head and neck squamous cell carcinoma, kidney chromophobe, liver hepatocellular carcinoma, lung adenocarcinoma, prostate adenocarcinoma, rectum adenocarcinoma, stomach adenocarcinoma, thyroid carcinoma and uterine corpus endometrial carcinoma. As transcriptional regulation of EPHB6 was suggested to be controlled by promoter methylation in breast cancer cell lines [16], we analyzed human cancer methylome data and found that EPHB6 is methylated in the promoter region in several malignancies, including breast cancer (FIG. 1B) as well as for example breast invasive carcinoma, cervical squamous cell carcinoma, colon adenocarcinoma, kidney renal clear cell carcinoma, lung adenocarcinoma, lung squamous cell carcinoma, pancreatic adenocarcinoma, prostate adenocarcinoma and rectum adenocarcinoma. Our assessment of TCGA data for immunohistochemistry-based breast cancer subtype classification revealed that EPHB6 is also significantly downregulated in patient samples, representing very heterogeneous and aggressive tumors of the TNBC group (FIG. 1C). Further computational analysis based on immunohistochemistry data confirmed that EPHB6 expression is reduced in at least 60% of the TNBC tumors, when compared to its mean expression level in the matching normal tissue [32, includes color figures].

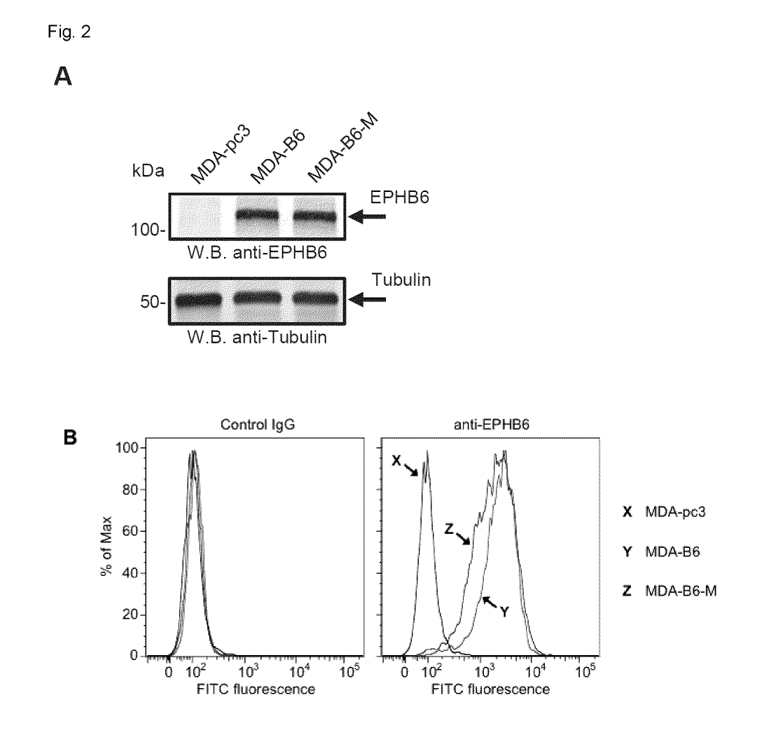

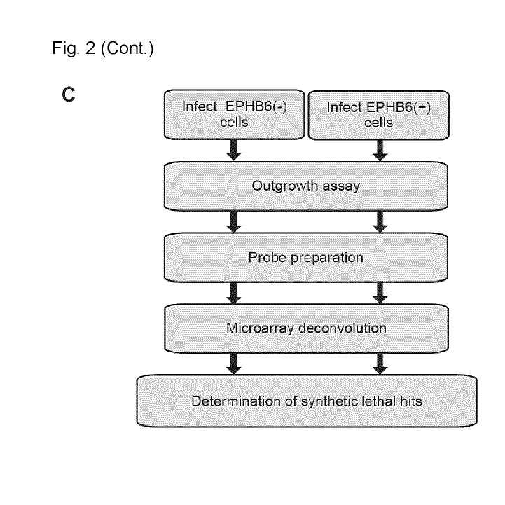

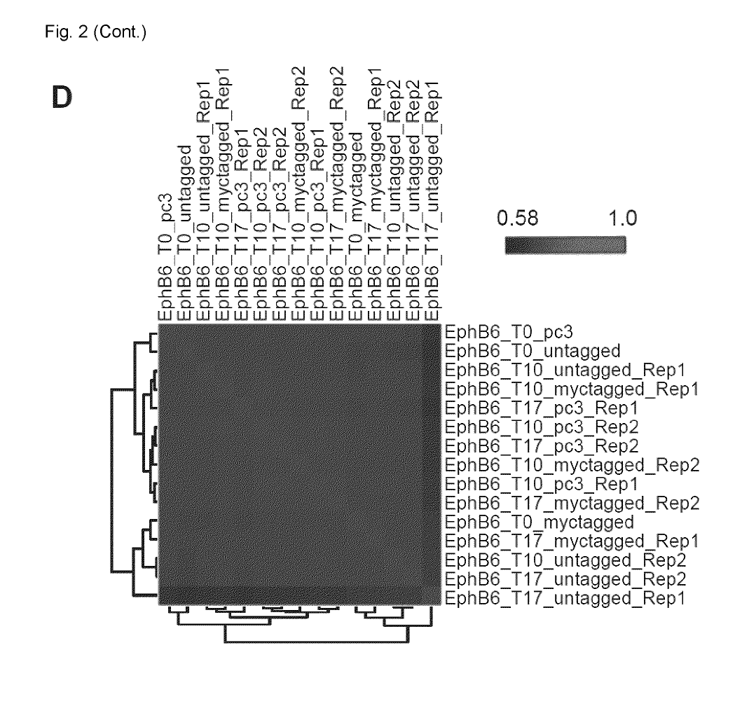

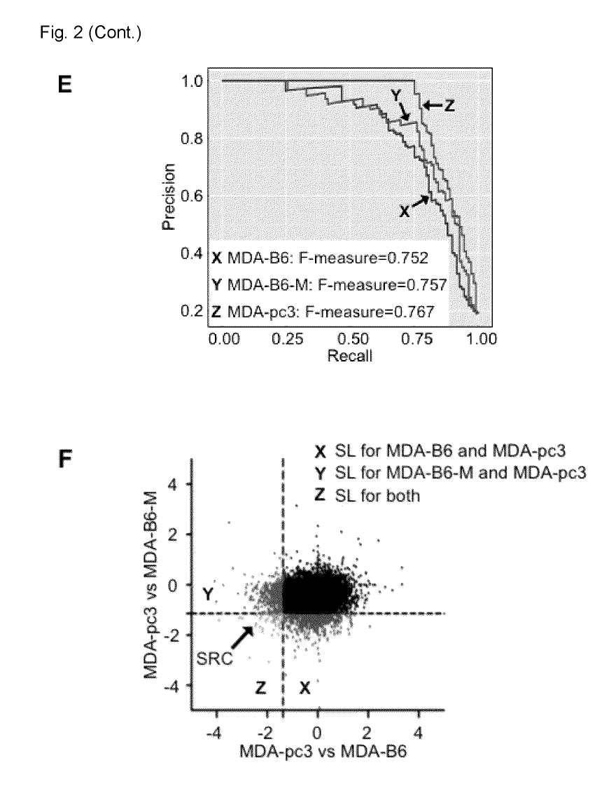

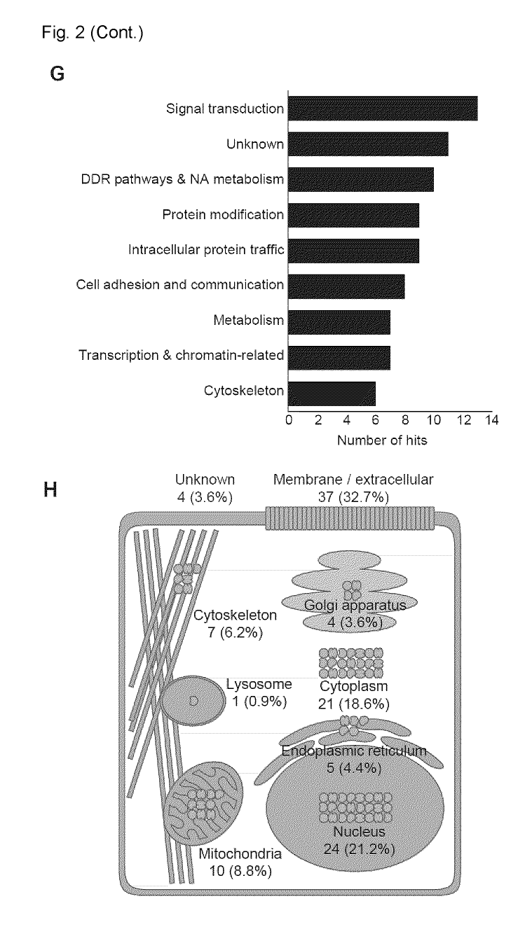

Since there is a strong need for a targeted therapy in TNBC, we conducted our SL screens in well-characterized TNBC cells, MDA-MB-231, that are often used in breast cancer-related research [21, 22]. MDA-MB-231 cells represent an excellent model for our investigation, as the ephb6 promoter is methylated and EPHB6 receptor expression is missing in these cells [15, 16]. In our experiments, we used cells with restored EPHB6 expression achieved by transfecting MDA-MB-231 cells with the pcDNA3 expression vector encoding wild-type EPHB6 (MDA-B6) or Myc-tagged EPHB6 (MDA-B6-M). Transfection with the empty vector was used as a control (MDA-pc3) (FIG. 2A). These cells were described in our previous work [19] which is incorporated herein by reference. Appropriate expression of the EPHB6 receptor on the surface of MDA-B6 and MDA-B6-M cells was confirmed by flow cytometry (FIG. 2B).

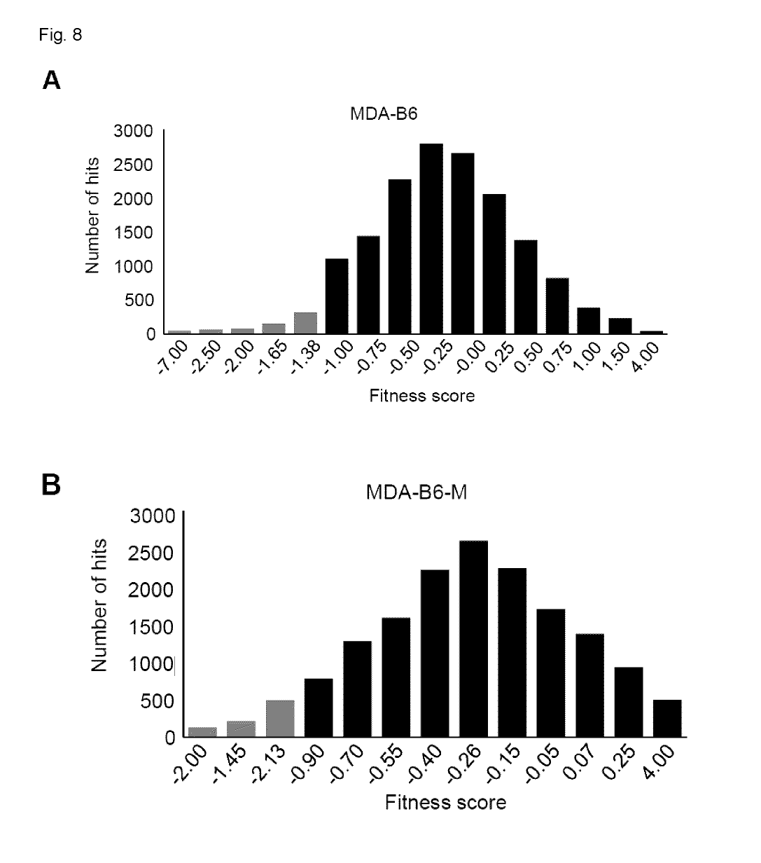

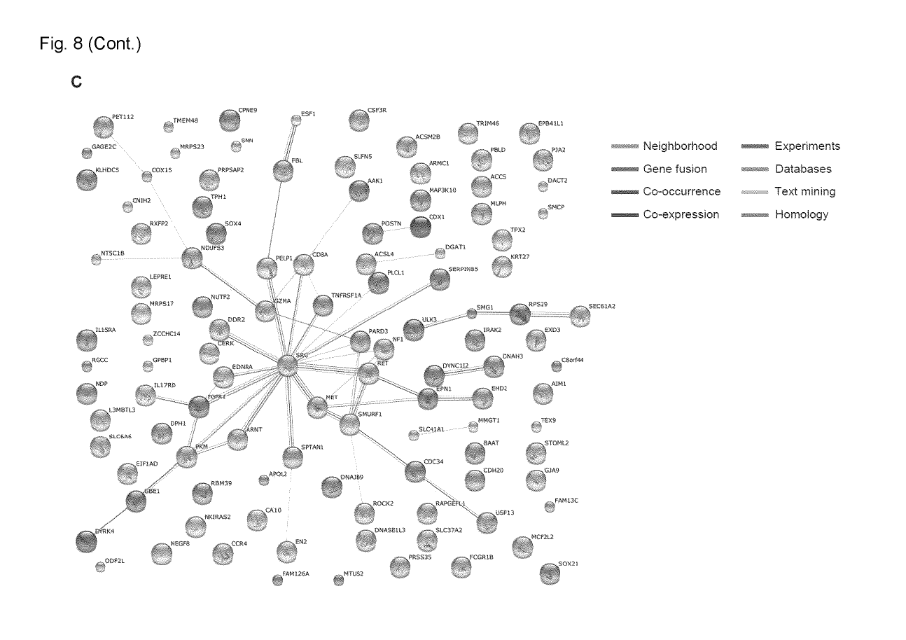

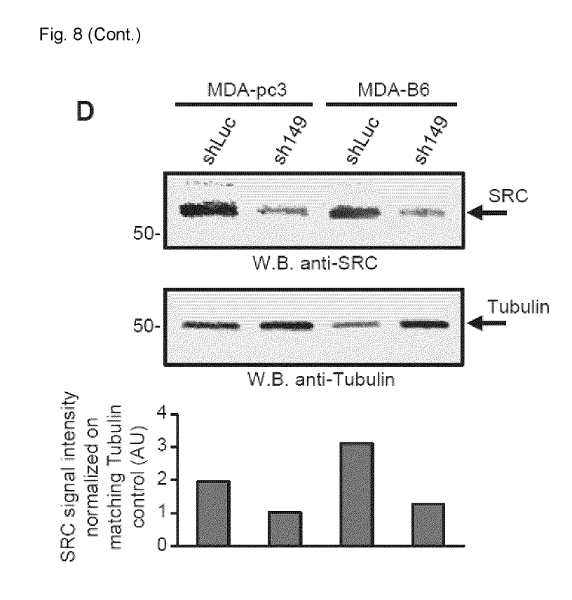

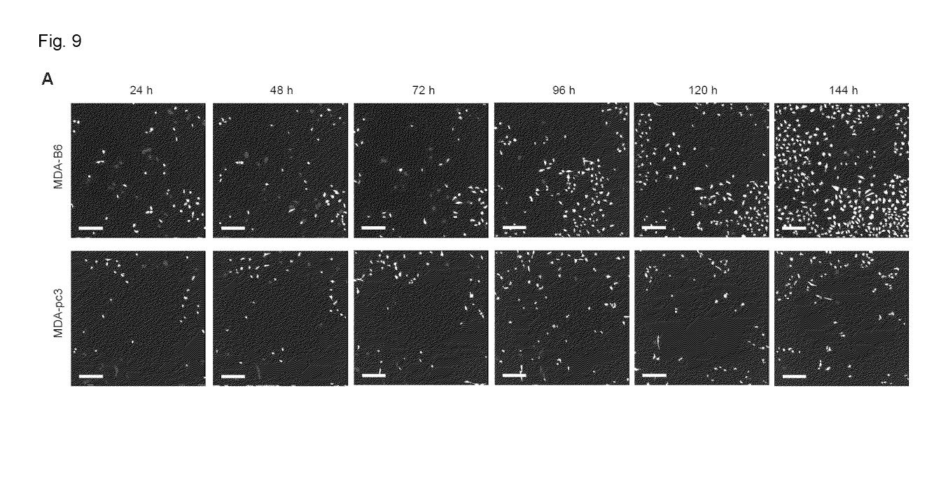

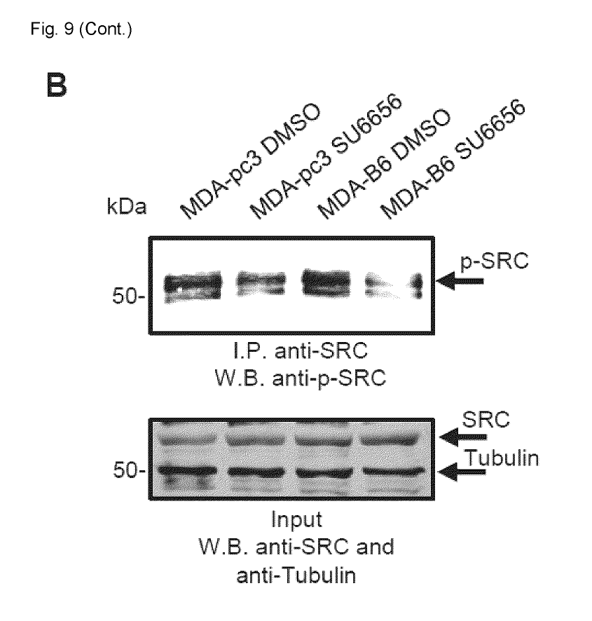

We used a lentiviral library that contains 90,000 unique viral hairpins representing 18,000 human genes to analyze thousands of di-genic interactions across three genetic backgrounds (MDA-pc3, MDA-B6 and MDA-B6-M) in duplicates. Following the infection of our cell lines, gene knockdowns that caused lethality were identified by the loss of associated barcodes on microarrays (FIG. 2C). The abundance of each shRNA was quantified by amplifying the hairpin sequences from the genomic DNA as a single mixture using vector-backbone directed universal primers. Specifically, shRNAs that dropped out in MDA-pc3, but not in MDA-B6 and MDA-B6-M populations are expected to target genes SL with EPHB6 deficiency. A correlation clustergram and the density plots of the three screens (MDA-pc3, MDA-B6, and MDA-B6-M) showed high reproducibility among the replicates (FIG. 2D). This is because genetic interactions are rare [23], and the relatively high correlation between the replicates at the different time points even after considering gene drop out suggests that a few highly sensitive SL interactions were detected in our screens (FIGS. 8A and 9B). Recently, a framework was developed for evaluating the quality of genome-scale lethality screens by assembling a reference set of essential genes [24]. If a high recall of these “gold standard” reference set of essential genes was achieved then the screen should be considered to be highly reliable [24]. Using this yardstick, we found that all three screens recorded excellent performance scores (F-measure>0.7) (FIG. 2E). In this analysis, the F-measure directly correlates with screen performance [24]. The trend of the hairpins that dropped specifically in EPHB6-deficient cells at different time points were computed as the Difference of Cumulative Change (DCC score) to identify top hits. The use of the top two hairpin scores per gene increased the confidence of the SL hits and allowed avoidance of possible off-target effects. As we used both Myc-tagged and untagged versions of EPHB6 in EPHB6-positive cells to compare against MDA-pc3, we determined the overlap between these two independent screens and identified 113 statistically significant overlapping hits (p<0.05) (FIG. 2F) (Table 1). This level of overlap reflects the genomic instability of breast cancer cells and a rate of potential false positive hits associated with large-scale screens. Therefore, considering hits identified in two independent cell lines increased the confidence in our analysis. Our approach identified a number of potential candidates that predominantly function in signal transduction (FIG. 2G), including molecules such as DDR2, SRC, ROCK2 and MET (Table 1). Consistent with the receptor functions of EPHB6, cellular localization analysis of the hits also revealed that a significant percentage of SL molecules spatially associated with the cell surface (FIG. 2H). Some of the hits were also associated with other cellular compartments, including nucleus and cytoplasm (FIG. 2H), which reflected the complexity of the network of EPHB6 functional interactions in cancer cells.

We next attempted to prioritize a potential target for further validation from our screen. To systematically select potential candidates for further investigation, we undertook a novel approach, where we coupled SL data with gene expression profiles. We rationalized that increased expression of a SL gene in EPHB6-deficient cells most likely represents an essential compensatory mechanism. To identify these essential molecules, we compiled the correlation between EPHB6 expression and expression of each SL hit that was identified in the pooled shRNA screen. This analysis was done across 25 different tumor types and specifically searched for a negative correlation between expression of EPHB6 and a SL gene. We found a non-receptor tyrosine kinase, SRC, to be clustered with a set of genes that mostly correlated negatively with EPHB6 expression (FIG. 3A). Consistent with this finding and in contrast to EPHB6 behavior, SRC is overexpressed in multiple malignancies (FIG. 3B). In addition, functional network analysis of all the 113 hits obtained from the screen using the STRING 10 database, which quantitatively integrates genomic and previously published interactions, positioned SRC as a hub with high connectivity to the rest of the hits (FIG. 8C). Overall, these observations identified SRC as a possible molecule for targeting EPHB6-deficient breast cancer cells.

To validate SL properties of SRC in EPHB6-deficient cells, we used an individual hairpin that efficiently silenced SRC expression (FIG. 8D). In agreement with our SL screen, we found that silencing of SRC with this hairpin caused a preferential suppression of EPHB6-deficient cells (FIG. 3C). To completely exclude the involvement of potential off-target effects of shRNA molecules, we chose to also validate this SL interaction using the CRISPR/Cas9-based system (FIG. 3D). Consistent with our earlier observations with SRC-silencing shRNAs, knockout of src with the CRISPR/Cas9 approach mostly affected EPHB6-defficient MDA-pc3 cells and produced only a limited effect on MDA-B6, thus further confirming the SL interaction between EPHB6 and SRC (FIGS. 3E-3G and 9A).

Synthetic lethal interaction between the SRC kinase and EPHB6 may be targeted by small molecule inhibitors in TNBC cells

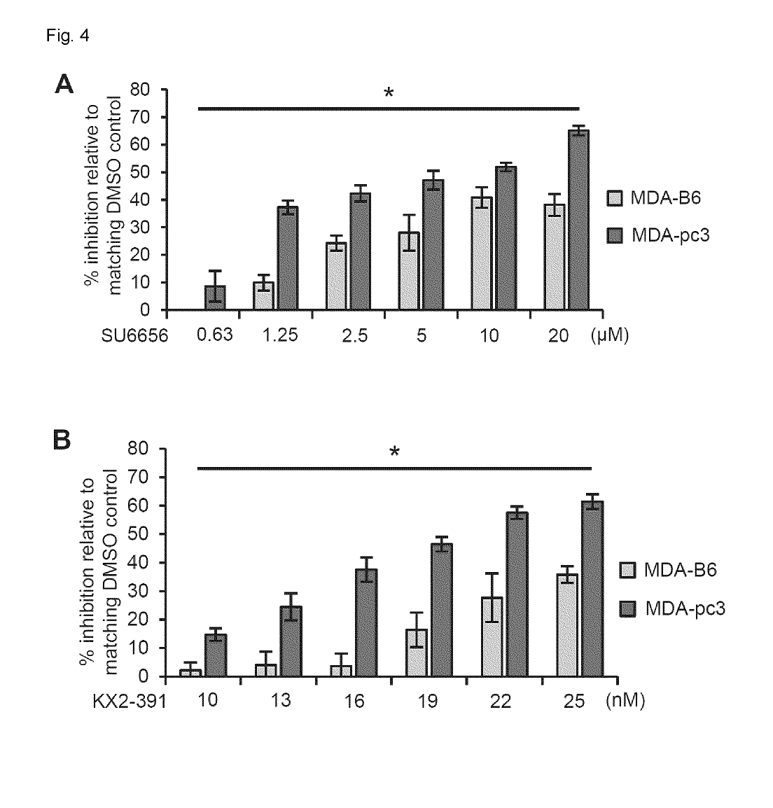

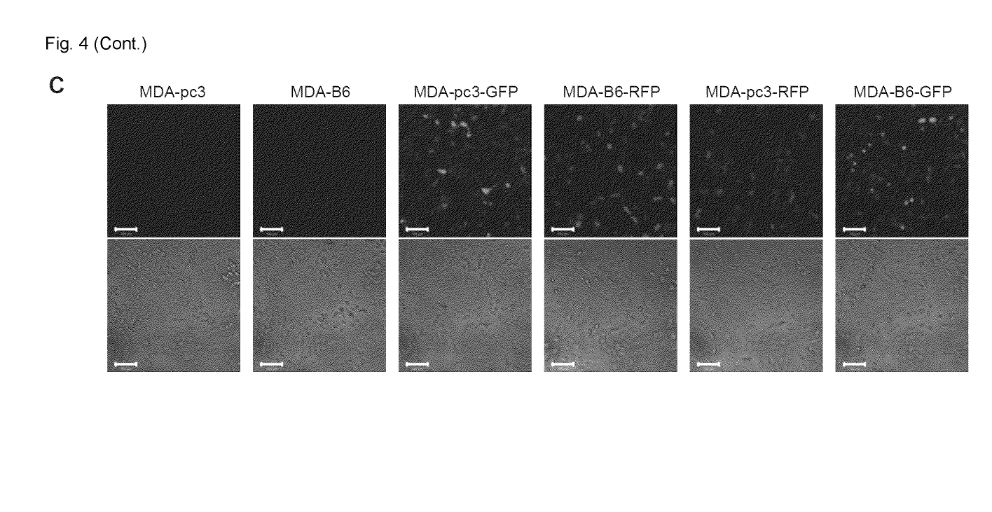

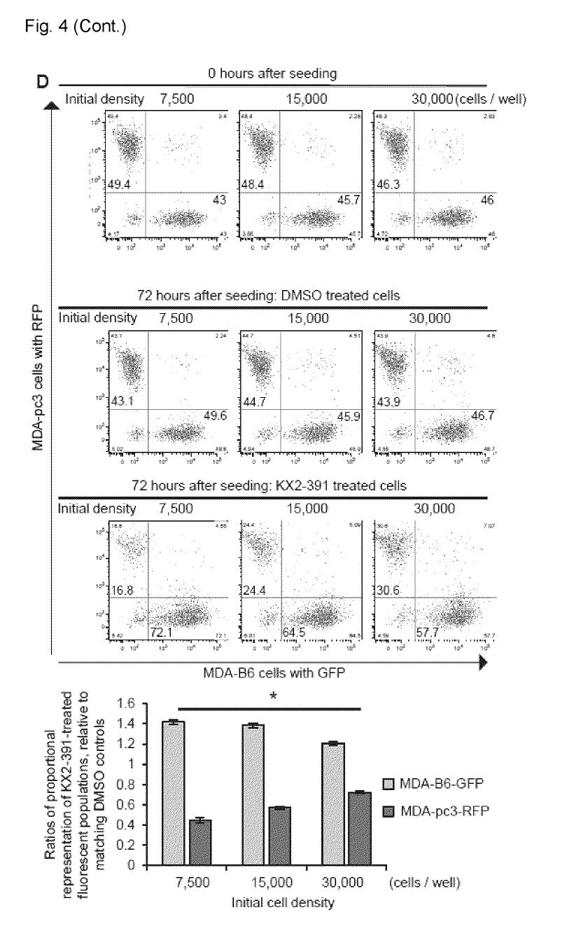

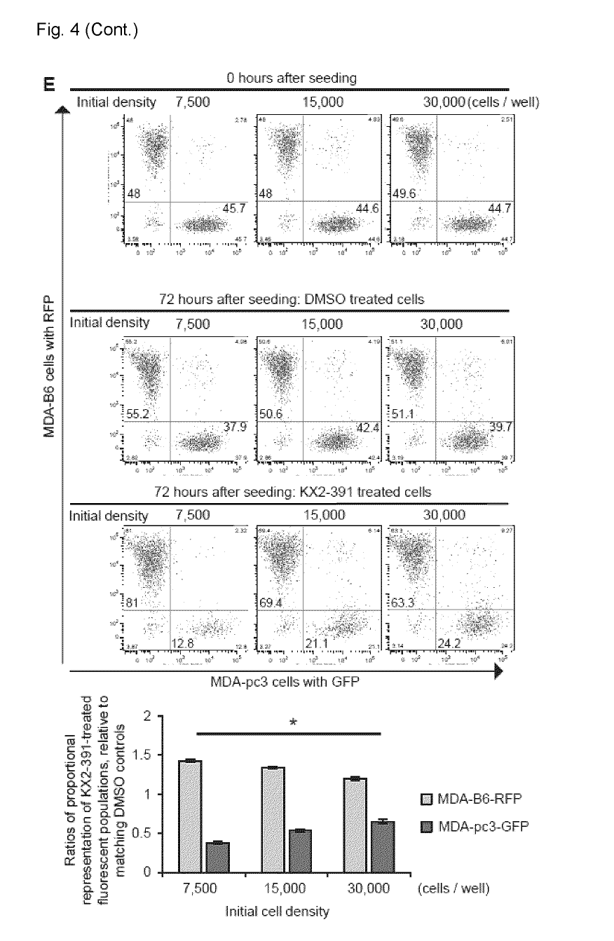

As SRC plays an important role in breast cancer progression and several SRC inhibitors are already being tested in breast cancer clinical trials [25], we used SRC inhibitors to further assess its SL properties. To model the SL interaction observed between SRC and EPHB6 by chemical genetics, we treated MDA-pc3 and MDA-B6 cells with increasing concentrations of an SRC inhibitor, SU6656. Consistent with the effects of the SRC-targeting shRNA or src knockout (FIG. 3C and 3F), application of SU6656 preferentially suppressed EPHB6-deficient MDA-pc3 cells (FIG. 4A). Another SRC inhibitor, KX2-391, has been tested in phase II clinical trials for prostate cancer treatment, where it showed a relatively modest effect [26]. KX2-391 is currently also being tested for breast cancer treatment (NCT01764087) and our finding of the SL relationship between EPHB6 and SRC indicated that KX2-391 treatment may work more efficiently if applied specifically to EPHB6-deficient TNBC cells. To assess this possibility, we incubated MDA-pc3 and MDA-B6 cells with this inhibitor or matching solvent control. In similarity to SU6656 action, KX2-391 caused significantly stronger suppression of EPHB6-deficent cells (FIG. 4B), suggesting that KX2-391 treatment may indeed potentially benefit from a more personalized approach, where it would be applied exclusively to EPHB6-deficient tumors. This observation was further confirmed in experiments with co-cultured MDA-B6 and MDA-pc3 cells, which allowed us to exclude any influence of potential differences in tissue culture conditions on experimental outcomes. In this model, EPHB6-defficient and EPHB6-expressing cells were stably transduced with a lentiviral vector expressing green or red fluorescent proteins (FIG. 4C). Cells were mixed, co-seeded in equal numbers, treated with KX2-391 or solvent control and cell suppression was monitored by flow cytometry. These experiments also clearly showed that EPHB6-deficient cells are much less resistant to SRC inhibition (FIGS. 4D and 4E).

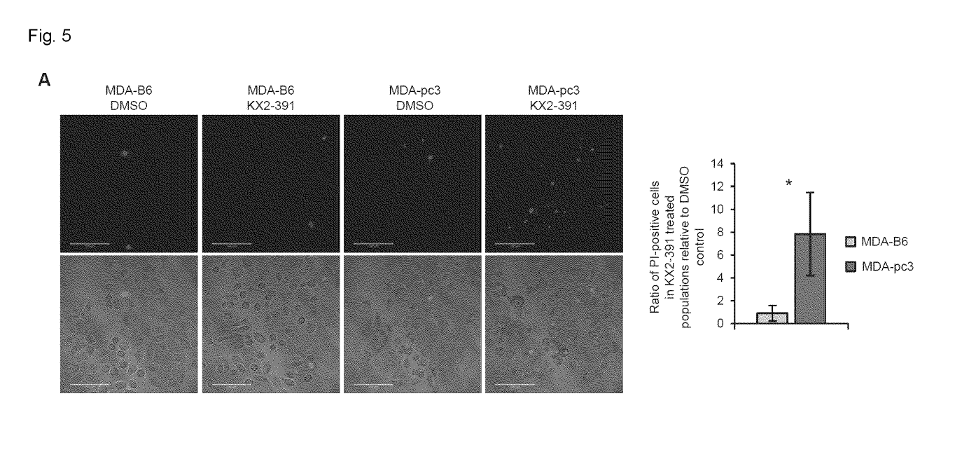

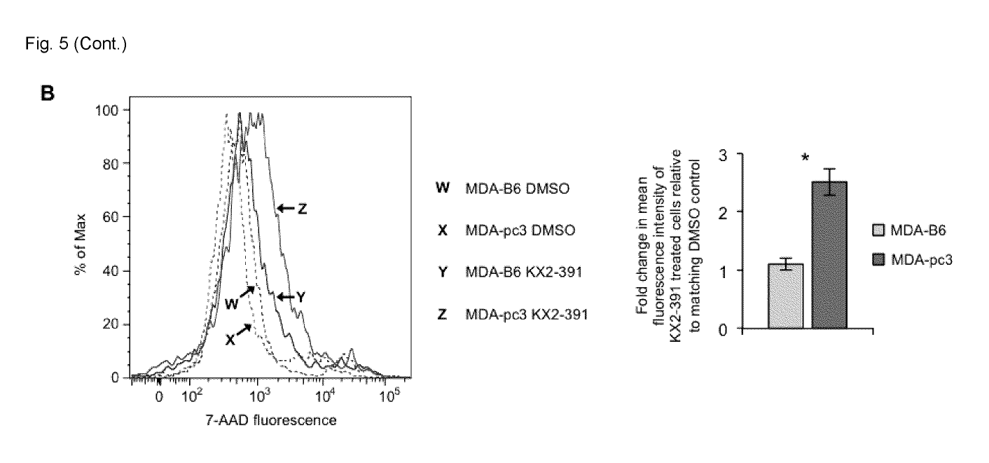

To examine if preferential suppression of EPHB6-defficient cells observed in our experiments is associated with more efficient cell killing, we exposed KX2-391-treated cultures to Propidium Iodide or 7-AAD compounds that stain nonviable cells only. Both approaches revealed that KX2-391 is more efficient in inducing cell death, when the EPHB6 receptor is not expressed (FIGS. 5A and 5B).

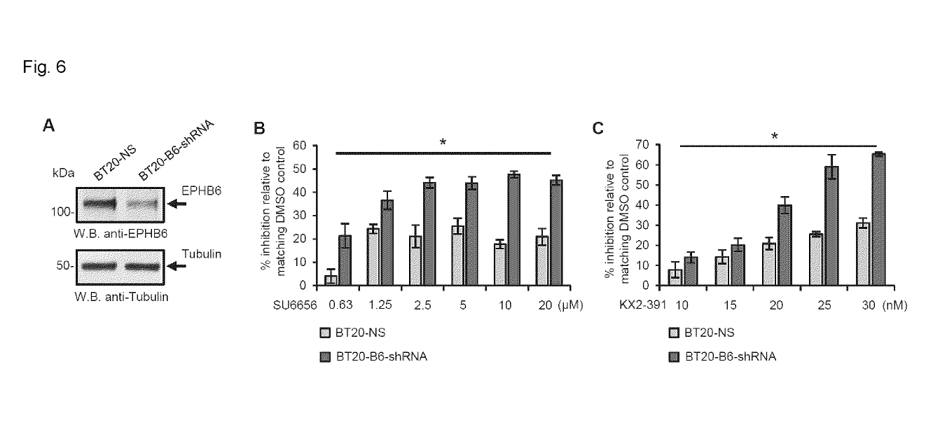

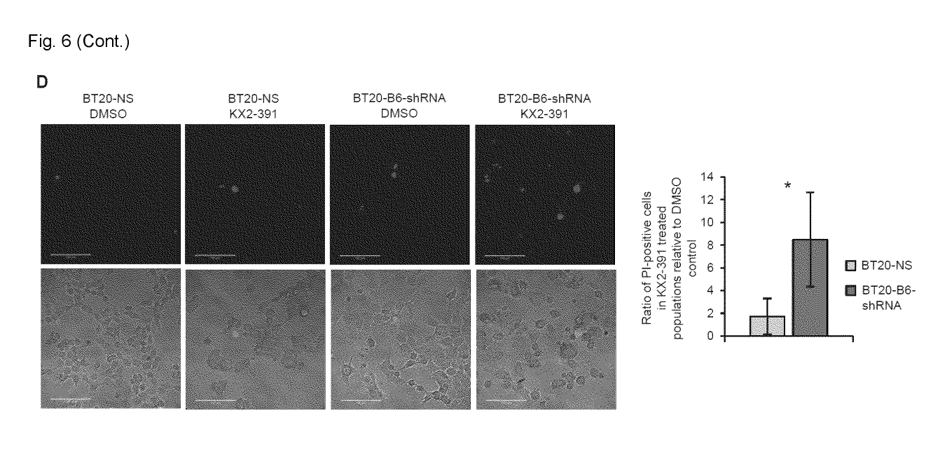

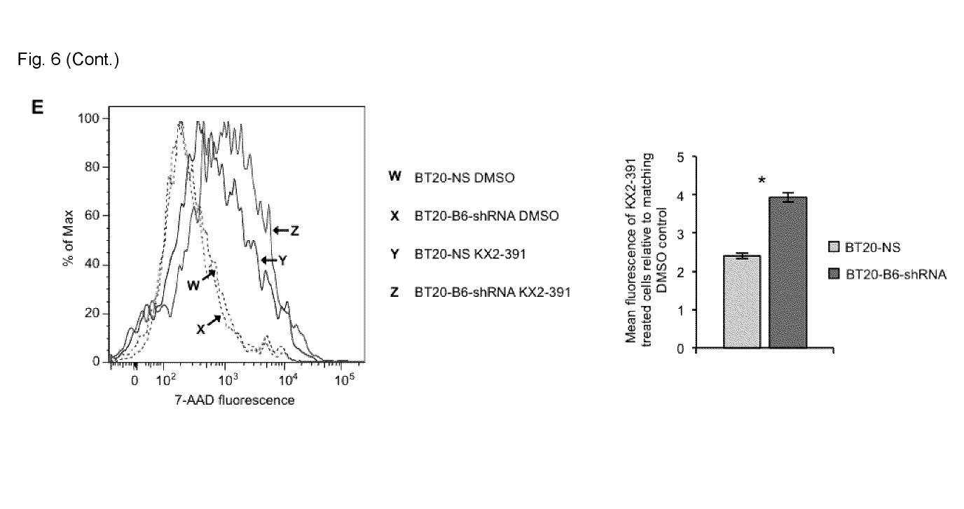

Importantly, the SL interaction between EPHB6 and SRC observed in our work was not restricted to MDA-MB-231 cells, since silencing of EPHB6 expression in another TNBC cell line, BT-20, strongly increased their suppression by both SU6656 and KX2-391 (FIGS. 6A-6E).

Despite the SL relation between EPHB6 and SRC that we observed in our work, EPHB6 did not affect SRC inhibition, as SRC was efficiently inhibited by SU6656 in MDA-B6 cells (FIG. 9B). These observations suggest that EPHB6 makes TNBC cells more resistant to SRC inhibitors, not by interfering with their direct effects on SRC activity, but most likely by compensating for the loss of SRC action in cellular responses controlled by this molecule.

The EPHB6-SRC synthetic lethality enhances suppression of TNBC tumors

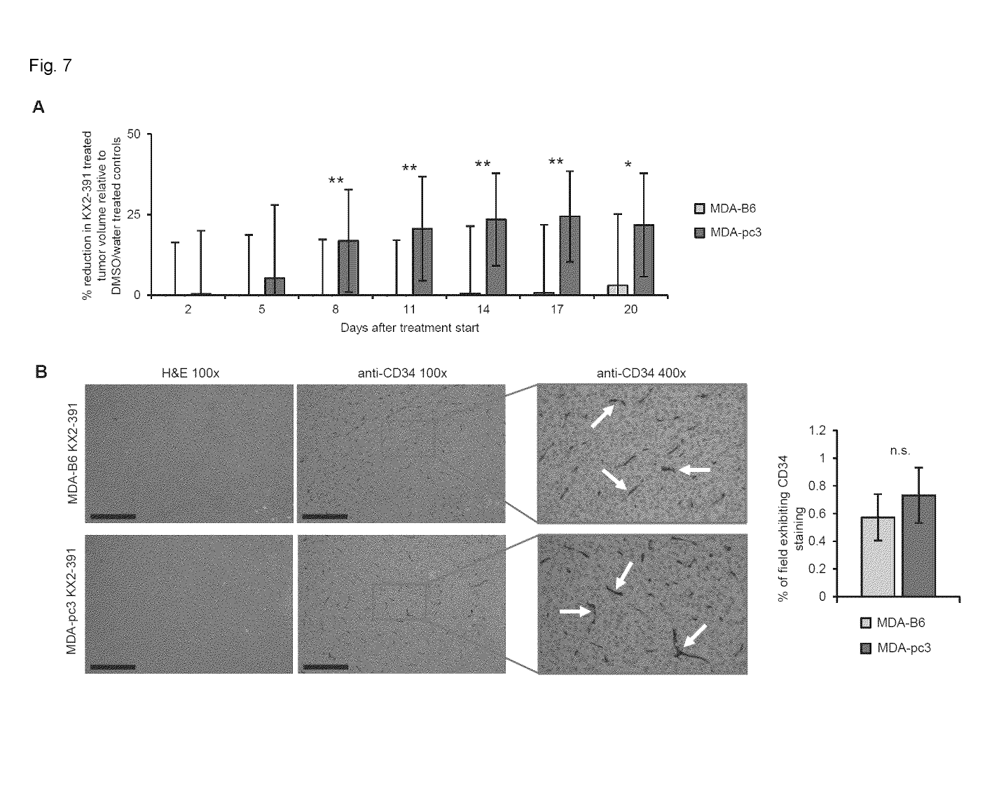

As SRC inhibitors are being actively evaluated in breast cancer clinical trials [25], our findings strongly suggested that the SL interaction between EPHB6 and SRC might be used to target TNBC tumors. To test this, we produced TNBC tumors in experimental animals by injecting MDA-pc3 and MDA-B6 cells in mammary fat pad regions of immunodeficient female NOD.Cg-Prkdcscid II2rgtm1Wjl/SzJ (NOD-SCID) mice. Treatment of these animals with KX2-391 was initiated when tumors reached a detectable size and was carried on until the mice had to be eliminated in accordance with the guidelines established by the University of Saskatchewan Animal Research Ethics Board. Excitingly, these experiments revealed that the KX2-391 therapy, indeed, more efficiently suppresses growth of EPHB6-deficient TNBC tumors (FIG. 7A). Staining for a blood vessel marker, CD34, has not shown any differences in neovascularization of KX2-391-treated EPHB6-positive or EPHB6-negative tumors, confirming that the observed lower resistance of EPHB6-deficient tumors was not due to the preferential suppression of blood vessel formation, but because of the higher sensitivity of EPHB6-deficient TNBC cells (FIG. 7B).

### DISCUSSION

Triple-negative breast tumors represent a breast cancer subtype that is characterized by the lack of estrogen receptor (ER) and progesterone receptor (PR) expression and does not overexpress the HER2 receptor. Triple-negative breast cancer (TNBC) is associated with a very high rate of patient mortality due to the complete absence of targeted therapies and there is an active search for efficient therapeutic targets that would allow treatment personalization in TNBC tumors [20]. Here, a genome-wide shRNA-based screen and a xenograft model of human TNBC were used to assess a possibility that EPHB6 deficiency may be targeted in TNBC by the SL approach and examine if SL may assist in personalizing TNBC therapy.

SL interactions have opened a new avenue for developing targeted therapies and personalized medicine. For example, at least three clinical trials have been initiated using EGFR and BRAF inhibitors within three years after the SL relation between EGFR and BRAF has been identified [30] (NCT01791309; NCT01750918; NCT01719380). This rapid progress into clinical trials is triggered by selective focusing on well-studied targets with the FDA approved inhibitors. The genome-wide SL screens discussed here revealed a novel genetic interaction between the SRC kinase and EPHB6 in TNBC cells. Moreover, network assessment directly indicated that SRC is a central player with a high connectivity. Our expression analysis also showed that SRC clusters with the genes that negatively correlate with EPHB6 expression in various tumors. This indirectly suggested that SRC overexpression might act as an essential compensatory mechanism for the loss of EPHB6 in cancer cells, indicating that the SL interaction of EPHB6 and SRC may represent a promising therapeutic target. The SRC kinase inhibitor, KX2-391, is already being tested in clinical trials and our investigation provides a new rationale for the selective use of KX2-391 in patients that have lost expression of the EPHB6 receptor in their tumors. The relevance of this finding is further supported by recent unfortunate observations, revealing that although SRC is frequently overexpressed in cancer, in some clinical trials randomly applied SRC inhibition produced limited positive effects on cancer patients [26]. Our report of the SL relation between EPHB6 and SRC may help to overcome this problem, and improve the efficiency of SRC inhibiting approaches in cancer therapy by showing that treatment with SRC inhibitors should be personalized, and mostly applied to patients with reduced or missing EPHB6 expression. In this context, it is important that our analysis confirmed that the SL interaction between SRC and EPHB6 can be efficiently targeted by small molecule SRC inhibitors and revealed that EPHB6-deficient TNBC cells are, indeed, much more sensitive to these compounds. Our experimental data suggest that EPHB6 does not protect SRC from inhibition and we suspect that EPHB6 most probably acts by partially compensating for the loss of the biological functions of the SRC kinase. This also explains well the ability of EPHB6 to protect cancer cells from shRNA-induced silencing of SRC or src knockout observed in our work. This model fits a classical definition of a SL interaction [31] and provides a rational for a limited effectiveness of SRC-inhibiting therapy currently observed in some cancer patients [26].

Consistent with the higher sensitivity of EPHB6-negative TNBC cells to SRC inhibition, an FDA-approved SRC kinase inhibitor, KX2-391, proved to be significantly more effective in suppressing EPHB6-deficient TNBC tumors, when compared to its effect on matching tumors with restored EPHB6 expression. These findings are of a potential practical importance, as our work reveals that although EPHB6 expression is overall downregulated in TNBC, it appears to be better preserved in a certain portion of TNBC tumors (FIG. 1C). Our observations indicate that in this situation, EPHB6 may be efficiently used as a biomarker for selecting exclusively EPHB6-deficient TNBC tumors for the treatment with SRC inhibitors, while re-directing patients with high EPHB6 expression in their tumors for more appropriate therapeutic options. Such a personalized approach is likely to assure successful utilization of SRC-inhibiting therapies and would also benefit patients with EPHB6-positive TNBC by preventing their involvement in ineffective treatment protocols. This of course would require a further evaluation of EPHB6 function in freshly obtained tumor samples. Our model may also potentially be applicable to multiple other tumor types, where EPHB6 expression is reduced according to previously published observations [8-14] and according to our findings reported here.

### Example 3

EPBH6-MET SL interaction and preferential suppression of MET inhibitor in EPBH6-deficient TNBC cells

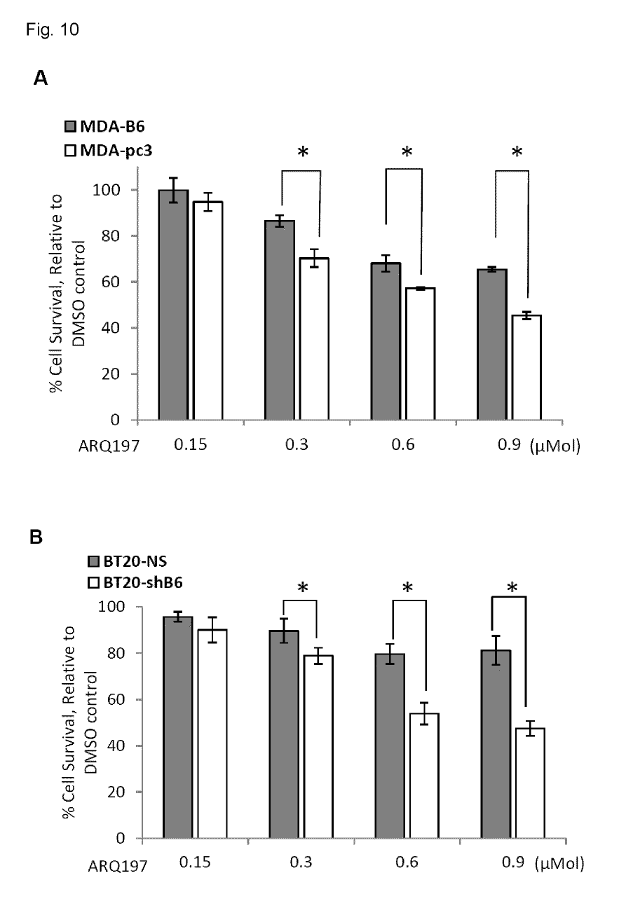

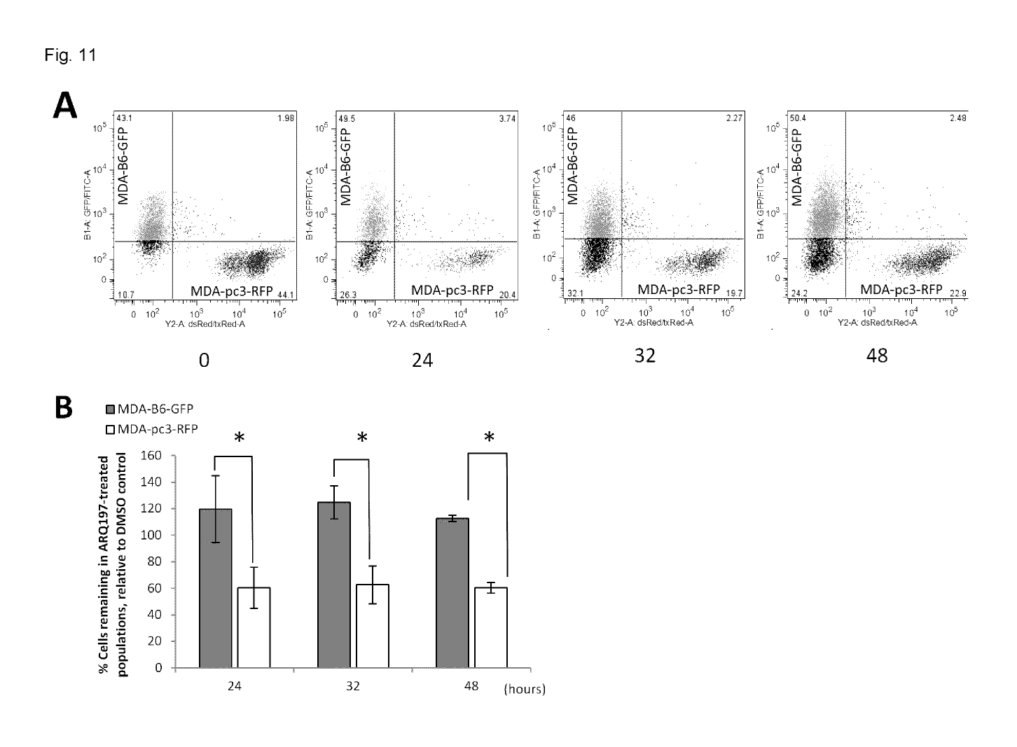

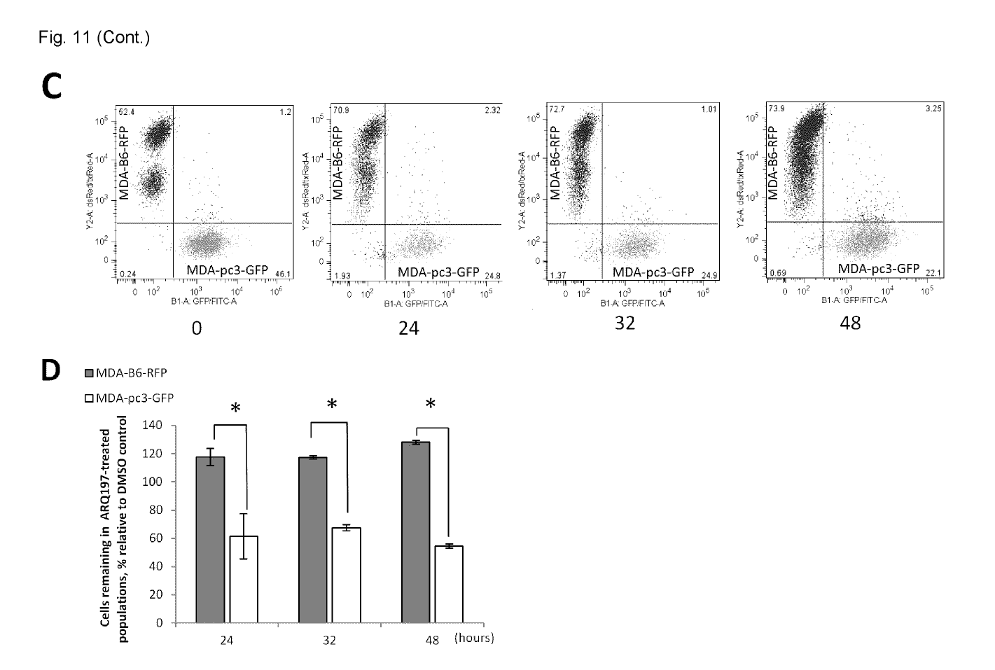

As mentioned above, a number of potential candidates that predominantly function in signal transduction (FIG. 2G), including DDR2, SRC, ROCK2 and MET (Table 1), were identified. Further testing with a MET inhibitor, ARQ197, was conducted to evaluate its SL properties. MDA-B6 and MDA-pc3 cells, as well as BT20-NS and BT20-shB6 cells, obtained using a method similar as described above in Example 1, were treated with increasing concentrations of ARQ197. EPHB6-deficient MDA-pc3 cells treated with ARQ197 were found to have decreased cell survival compared to MDA-B6 cells transfected with EPBH6 (FIG. 10A). Similarly, BT20-shB6 cells transduced with EPBH6-targeting shRNA had decreased cell survival compared to BT-20 cells transduced with non-silencing shRNA (FIG. 10B). In addition, as shown in FIG. 11, treatment with ARQ197 preferentially suppressed EPPBH6-deficient MDA-B6 cells, when co-cultured colour-coded MDA-pc3 and AMDA-B6 cells were used.

These findings indicate that MET inhibitors such as ARQ197 may represent a suitable treatment or be included in a suitable treatment for EPBH6-deficient tumors and that EPHB6 may be efficiently used as a biomarker for selecting exclusively EPHB6-deficient TNBC tumors for the treatment with MET inhibitors.

### Example 4

Patients having or suspected of having cancer can be treated according to the following method. A biological sample is first obtained from the patient. The biological sample can be for example a tumor sample such as a tumor sample obtained from a biopsy. The level of EPHB6 receptor in the biological sample is determined for example by measuring the level of EPHB6 receptor protein or mRNA, optionally by a RT-PCR method. The level of EPHB6 receptor can also be determined using antibody based methods. The level of EPHB6 receptor can also be determined by measuring EPHB6 promoter methylation. When the EPHB6 receptor levels are deficient or below a selected threshold, the patient will be identified as being suitable for treatment with an inhibitor of a Table 1 molecule, and will be administered an effective amount of the inhibitor of a Table 1 molecule. For example, the inhibitor is a SRC kinase inhibitor or a MET kinase inhibitor. When the EPHB6 receptor levels are comparable or increased compared to a control such as for example adjacent normal tissue, the patient will be identified as not suitable for treatment with an inhibitor of a Table 1 molecule and will instead be treated with an alternate therapeutic.

TABLE 1
List of EPHB6 synthetic lethal interactions

| Gene ID | Gene Symbol |
|---|---|
| 22848 | AAK1 |
| 84680 | ACCS |
| 2182 | ACSL4 |
| 348158 | ACSM2B |
| 202 | AIM1 |
| 23780 | APOL2 |
| 55156 | ARMC1 |
| 405 | ARNT |
| 570 | BAAT |
| 28984 | C13orf15 |
| 56260 | C8orf44 |
| 56934 | CA10 |
| 1233 | CCR4 |
| 925 | CD8A |
| 997 | CDC34 |
| 28316 | CDH20 |
| 1044 | CDX1 |
| 64781 | CERK |
| 254263 | CNIH2 |
| 1355 | COX15 |
| 1348 | COX7AP2 |
| 151835 | CPNE9 |
| 1441 | CSF3R |
| 168002 | DACT2 |
| 4921 | DDR2 |
| 8694 | DGAT1 |
| 55567 | DNAH3 |
| 4189 | DNAJB9 |
| 1776 | DNASE1L3 |
| 1801 | DPH1 |
| 1781 | DYNC1I2 |
| 8798 | DYRK4 |
| 1909 | EDNRA |
| 30846 | EHD2 |
| 84285 | EIF1AD |
| 2020 | EN2 |
| 2036 | EPB41L1 |
| 29924 | EPN1 |
| 51575 | ESF1 |
| 54932 | EXD3 |
| 84668 | FAM126A |
| 220965 | FAM13C |
| 2091 | FBL |
| 2210 | FCGR1B |
| 2260 | FGFR1 |
| 2574 | GAGE2C |
| 2632 | GBE1 |
| 81025 | GJA9 |
| 65056 | GPBP1 |
| 3001 | GZMA |
| 3601 | IL15RA |
| 54756 | IL17RD |
| 3656 | IRAK2 |
| 23281 | KIAA0774 |
| 57542 | KLHDC5 |
| 342574 | KRT27 |
| 84456 | L3MBTL3 |
| 64175 | LEPRE1 |
| 4294 | MAP3K10 |
| 23101 | MCF2L2 |
| 1954 | MEGF8 |
| 4233 | MET |
| 79083 | MLPH |
| 93380 | MMGT1 |
| 51373 | MRPS17 |
| 51649 | MRPS23 |
| 4693 | NDP |
| 4722 | NDUFS3 |
| 4763 | NF1 |
| 28511 | NKIRAS2 |
| 93034 | NT5C1B |
| 10204 | NUTF2 |
| 57489 | ODF2L |
| 56288 | PARD3 |
| 64081 | PBLD |
| 27043 | PELP1 |
| 5188 | PET112L |
| 9867 | PJA2 |
| 5315 | PKM2 |
| 5334 | PLCL1 |
| 10631 | POSTN |
| 5636 | PRPSAP2 |
| 167681 | PRSS35 |
| 51195 | RAPGEFL1 |
| 9584 | RBM39 |
| 5979 | RET |
| 9475 | ROCK2 |
| 6235 | RPS29 |
| 122042 | RXFP2 |
| 55176 | SEC61A2 |
| 5268 | SERPINB5 |
| 219855 | SLC37A2 |
| 254428 | SLC41A1 |
| 6533 | SLC6A6 |
| 162394 | SLFN5 |
| 4184 | SMCP |
| 23049 | SMG1 |
| 57154 | SMURF1 |
| 8303 | SNN |
| 11166 | SOX21 |
| 6659 | SOX4 |
| 6709 | SPTAN1 |
| 6714 | SRC |
| 30968 | STOML2 |
| 374618 | TEX9 |
| 55706 | TMEM48 |
| 7132 | TNFRSF1A |
| 7166 | TPH1 |
| 22974 | TPX2 |
| 80128 | TRIM46 |
| 25989 | ULK3 |
| 8975 | USP13 |
| 23174 | ZCCHC14 |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Table or elsewhere, are incorporated by reference in its entirely.

### CITATIONS FOR REFERENCES REFERRED TO IN THE SPECIFICATION

1. Arnedos M, Vicier C, Loi S, Lefebvre C, Michiels S, Bonnefoi H and Andre F. Precision medicine for metastatic breast cancer-limitations and solutions. Nat Rev Clin Oncol. 2015; 12(12):693-704.

2. Paul J M, Templeton S D, Baharani A, Freywald A and Vizeacoumar F J. Building high-resolution synthetic lethal networks: a ‘Google map’ of the cancer cell. Trends Mol Med. 2014; 20(12):704-715.

3. Vizeacoumar F J, Arnold R, Vizeacoumar F S, Chandrashekhar M, Buzina A, Young J T, Kwan J H, Sayad A, Mero P, Lawo S, Tanaka H, Brown K R, Baryshnikova A, Mak A B, Fedyshyn Y, Wang Y, et al. A negative genetic interaction map in isogenic cancer cell lines reveals cancer cell vulnerabilities. Mol Syst Biol. 2013; 9:696.

4. Gurniak C B and Berg L J. A new member of the Eph family of receptors that lacks protein tyrosine kinase activity. Oncogene. 1996; 13(4):777-786.

5. Pasquale E B. Eph receptors and ephrins in cancer: bidirectional signalling and beyond. Nat Rev Cancer. 2010; 10(3):165-180.

6. Lisabeth E M, Falivelli G and Pasquale E B. Eph receptor signaling and ephrins. Cold Spring Harb Perspect Biol. 2013; 5(9).

7. Chen J. Regulation of tumor initiation and metastatic progression by Eph receptor tyrosine kinases. Adv Cancer Res. 2012; 114:1-20.

8. Muller-Tidow C, Diederichs S, Bulk E, Pohle T, Steffen B, Schwable J, Plewka S, Thomas M, Metzger R, Schneider P M, Brandts C H, Berdel W E and Serve H. Identification of metastasis-associated receptor tyrosine kinases in non-small cell lung cancer. Cancer Res. 2005; 65(5):1778-1782.

9. Hafner C, Bataille F, Meyer S, Becker B, Roesch A, Landthaler M and Vogt T. Loss of EphB6 expression in metastatic melanoma. Int J Oncol. 2003; 23(6):1553-1559.

10. Mohamed E R, Noguchi M, Hamed A R, Eldahshoury M Z, Hammady A R, Salem E E and Itoh K. Reduced expression of erythropoietin-producing hepatocyte B6 receptor tyrosine kinase in prostate cancer. Oncol Lett. 2015; 9(4):1672-1676.

11. Gu Y, Li F, Qian N, Chen X, Wang H and Wang J. Expression of EphB6 in ovarian serous carcinoma is associated with grade, TNM stage and survival. J Clin Pathol. 2015.

12. Liersch-Lohn B, Slavova N, Buhr H J and Bennani-Baiti I M. Differential protein expression and oncogenic gene network link tyrosine kinase ephrin B4 receptor to aggressive gastric and gastroesophageal junction cancers. Int J Cancer. 2015.

13. Tang X X, Evans A E, Zhao H, Cnaan A, London W, Cohn S L, Brodeur G M and Ikegaki N. High-level expression of EPHB6, EFNB2, and EFNB3 is associated with low tumor stage and high TrkA expression in human neuroblastomas. Clin Cancer Res. 1999; 5(6):1491-1496.

14. Tang X X, Zhao H, Robinson M E, Cnaan A, London W, Cohn S L, Cheung N K, Brodeur G M, Evans A E and Ikegaki N. Prognostic significance of EPHB6, EFNB2, and EFNB3 expressions in neuroblastoma. Med Pediatr Oncol. 2000; 35(6):656-658.

15. Fox B P and Kandpal R P. Invasiveness of breast carcinoma cells and transcript profile: Eph receptors and ephrin ligands as molecular markers of potential diagnostic and prognostic application. Biochem Biophys Res Commun. 2004; 318(4):882-892.

16. Fox B P and Kandpal R P. Transcriptional silencing of EphB6 receptor tyrosine kinase in invasive breast carcinoma cells and detection of methylated promoter by methylation specific PCR. Biochem Biophys Res Commun. 2006; 340(1):268-276.

17. Yu J, Bulk E, Ji P, Hascher A, Tang M, Metzger R, Marra A, Serve H, Berdel W E, Wiewroth R, Koschmieder S and Muller-Tidow C. The EPHB6 receptor tyrosine kinase is a metastasis suppressor that is frequently silenced by promoter DNA hypermethylation in non-small cell lung cancer. Clin Cancer Res. 2010; 16(8):2275-2283.

18. Bailey C M and Kulesa P M. Dynamic Interactions between Cancer Cells and the Embryonic Microenvironment Regulate Cell Invasion and Reveal EphB6 as a Metastasis Suppressor. Mol Cancer Res. 2014; 12(9):1303-1313.

19. Truitt L, Freywald T, DeCoteau J, Sharfe N and Freywald A. The EphB6 receptor cooperates with c-Cbl to regulate the behavior of breast cancer cells. Cancer Res. 2010; 70(3):1141-1153.

20. Mayer I A, Abramson V G, Lehmann B D and Pietenpol J A. New strategies for triple-negative breast cancer—deciphering the heterogeneity. Clin Cancer Res. 2014; 20(4):782-790.

21. Wang L, Zhao Z, Meyer M B, Saha S, Yu M, Guo A, Wisinski K B, Huang W, Cai W, Pike J W, Yuan M, Ahlquist P and Xu W. CARM1 methylates chromatin remodeling factor BAF155 to enhance tumor progression and metastasis. Cancer Cell. 2014; 25(1):21-36.

22. Zhang K, Corsa C A, Ponik S M, Prior J L, Piwnica-Worms D, Eliceiri K W, Keely P J and Longmore G D. The collagen receptor discoidin domain receptor 2 stabilizes SNAIL1 to facilitate breast cancer metastasis. Nat Cell Biol. 2013; 15(6):677-687.

23. Boone C, Bussey H and Andrews B J. Exploring genetic interactions and networks with yeast. Nat Rev Genet. 2007; 8(6):437-449.

24. Hart T, Brown K R, Sircoulomb F, Rottapel R and Moffat J. Measuring error rates in genomic perturbation screens: gold standards for human functional genomics. Mol Syst Biol. 2014; 10:733.

25. Hosford S R and Miller T W. Clinical potential of novel therapeutic targets in breast cancer: CDK4/6, Src, JAK/STAT, PARP, HDAC, and PI3K/AKT/mTOR pathways. Pharmgenomics Pers Med. 2014; 7:203-215.

26. Antonarakis E S, Heath E I, Posadas E M, Yu E Y, Harrison M R, Bruce J Y, Cho S Y, Wilding G E, Fetterly G J, Hangauer D G, Kwan M F, Dyster L M and Carducci M A. A phase 2 study of KX2-391, an oral inhibitor of Src kinase and tubulin polymerization, in men with bone-metastatic castration-resistant prostate cancer. Cancer Chemother Pharmacol. 2013; 71(4):883-892.

27. Martin G S. The hunting of the Src. Nat Rev Mol Cell Biol. 2001; 2(6):467-475.

28. Yu J, Bulk E, Ji P, Hascher A, Koschmieder S, Berdel W E and Muller-Tidow C. The kinase defective EPHB6 receptor tyrosine kinase activates MAP kinase signaling in lung adenocarcinoma. Int J Oncol. 2009; 35(1):175-179.

29. Bhushan L, Tavitian N, Dey D, Tumur Z, Parsa C and Kandpal R P. Modulation of liver-intestine cadherin (Cadherin 17) expression, ERK phosphorylation and WNT signaling in EPHB6 receptor-expressing MDA-MB-231 cells. Cancer Genomics Proteomics. 2014; 11(5):239-249.

30. Prahallad A, Sun C, Huang S, Di Nicolantonio F, Salazar R, Zecchin D, Beijersbergen R L, Bardelli A and Bernards R. Unresponsiveness of colon cancer to BRAF(V600E) inhibition through feedback activation of EGFR. Nature. 2012; 483(7387):100-103.

31. Dixon S J, Costanzo M, Baryshnikova A, Andrews B and Boone C. Systematic mapping of genetic interaction networks. Annu Rev Genet. 2009; 43:601-625.

32. Paul et al. Targeting synthetic lethality between the SRC kinase and the EPHB6 receptor may benefit cancer treatment, 2016 Oncotarget, Vol. 7, No. 31.

1. A method of:
i) identifying a subject with a cancer eligible for treatment with an inhibitor of a Table 1 molecule, optionally a SRC kinase inhibitor or a MET kinase inhibitor, comprising testing a biological sample from the subject for a deficiency in EPHB6 receptor levels, optionally EPHB6 receptor polypeptide or transcript levels, wherein the subject is eligible for treatment with the inhibitor of a Table 1 molecule, optionally the SRC kinase inhibitor or the MET kinase inhibitor, if EPHB6 receptor levels in the biological sample are deficient; or
ii) treating a cancer in a subject comprising: administering an effective amount of an inhibitor of a Table 1 molecule, optionally a SRC kinase inhibitor or a MET kinase inhibitor, to a subject in need of such a treatment, wherein the subject in need of such treatment is a subject wherein the cancer is deficient for EPHB6 receptor levels optionally identified by evaluating EPHB6 receptor levels in a biological sample of a subject suspected from having from cancer, having cancer or being prone to having cancer, and wherein a deficiency in EPHB6 receptor levels in the biological sample, optionally compared to a control, indicates responsiveness of the subject to the inhibitor of a Table 1 molecule, optionally the SRC kinase inhibitor or the MET kinase inhibitor.

2. (canceled)

3. The method of claim 1, wherein the biological sample is a tumor sample or a biopsy.

4. The method of claim 1, wherein the cancer has a deficiency in EPHB6 receptor levels.

5. The method of claim 1 ii), wherein the method further comprises testing for a deficiency in EPHB6 receptor levels in a biological sample from the patient and administering a therapeutically effective amount of an inhibitor of a Table 1 molecule, optionally a SRC kinase inhibitor or a MET kinase inhibitor, to the patient if the biological sample tests positive for a deficiency in EPHB6 receptor levels.

6. The method of claim 1, wherein the deficiency in EPHB6 receptor levels is determined by measuring the level of EPHB6 receptor protein or mRNA.

7. The method of claim 6, wherein the mRNA level is detected by a RT-PCR method.

8. The method of claim 1, wherein the biological sample is deficient in EPHB6 receptor levels if the level is at least 20% decreased, at least 30% decreased, at least 40% decreased, at least 50% decreased, at least 60% decreased, at least 70% decreased, at least 80% decreased, at least 90% decreased or more relative to a control, normal tissue and/or normal cells.

9. The method of claim 1, wherein the deficiency in EPHB6 receptor levels is determined when the level is undetectable using a standard assay or below a selected threshold.

10. The method claim 1, wherein the deficiency in EPHB6 receptor levels is determined by determining EPHB6 promoter methylation.

11. A method of
i) personalizing treatment in a subject having or suspected of having cancer comprising measuring EPHB6 receptor levels in a biological sample obtained from the subject, comparing the measured EPHB6 receptor levels to a control, treating the subject with an inhibitor of a Table 1 molecule, optionally a SRC kinase inhibitor or a MET kinase inhibitor when the EPHB6 receptor levels are deficient, and otherwise treating the subject with an alternate treatment, for example when the EPHB6 receptor levels are comparable or increased compared to a control such as adjacent normal tissue; or
ii) selecting a therapeutic for a subject having or suspected of having cancer, the method comprising:
a) obtaining a biological sample from the subject,
b) measuring EPHB6 receptor levels in the biological sample, and
c) selecting an inhibitor of a Table 1 molecule, optionally a SRC kinase inhibitor or a MET kinase inhibitor, as the therapeutic when a deficiency in EPHB6 receptor levels is measured in the biological sample or selecting an alternate therapeutic, for example when the EPHB6 receptor levels are comparable or increased compared to a control such as adjacent normal tissue.

12. (canceled)

13. The method of claim 1, wherein the cancer is selected from breast cancer, including for example invasive breast cancer and/or triple negative breast cancer (TNBC), lung cancer, melanoma, prostate cancer, ovarian carcinoma, gastric cancer, colon cancer, neuroblastoma including aggressive neuroblastoma and from an EphB6-deficient cancer listed in FIG. 1.

14. The method of claim 1, wherein the SRC kinase inhibitor is selected from dasatinib, bosutinib (SKI-606), saracatinib (AZD530), SU6656, KX2-391 and/or posatinib (AP24534), PPI, PP2, Quercetin and/or pharmaceutically acceptable salts, solvates, and/or hydrates thereof.

15. The method of claim 1, wherein the MET kinase inhibitor is selected from tivantinib (ARQ197), K252a, SU11274, AM7, PHA-665752, PF-2341066, foretinib, SGX523, MP470, crizotinib, cabozantinib, and/or pharmaceutically acceptable salts, solvates, and/or hydrates thereof.

16-37. (canceled)

38. A screening assay, comprising:
contacting a control cancer cell sample with a test candidate;
contacting a second control cancer cell sample and a second test cancer cell sample with a known inhibitor of a Table 1 molecule, optionally a known SRC kinase inhibitor or a known MET kinase inhibitor;
contacting a test cancer cell sample deficient in EPHB6 receptor levels with the test candidate;
measuring an effect of the test candidate on the control cancer cell sample, on the test cancer cell sample and on the second control cancer cell sample;
comparing the effect of the test candidate on the control cancer cell sample and on the test cancer cell sample; and
identifying the test candidate as a putative inhibitor, optionally a putative SRC kinase inhibitor or a putative MET kinase inhibitor, when the effect measured is greater on the test cancer cell sample compared to the control cancer cell sample and the effect measured is at least comparable to the known inhibitor.

39. (canceled)

40. The screening assay of claim 38, wherein the effect measured is cell death and/or decreased in cell proliferation and the test candidate that induces cell death and/or inhibits cell proliferation, optionally by at least a comparable level to the known inhibitor, is identified as a putative inhibitor.

41. The screening assay of claim 38, wherein the control cancer cell sample is adjacent normal tissue or a non EPHB6 deficient cancer cell sample and the test cancer cell sample is a test tumor, the effect measured is tumor volume, and the test candidate that decreases the tumor volume and/or suppresses tumor growth, by at least a comparable level to the known inhibitor, is identified as a putative inhibitor.

42. The method of claim 11, wherein the cancer is selected from breast cancer, including for example invasive breast cancer and/or triple negative breast cancer (TNBC), lung cancer, melanoma, prostate cancer, ovarian carcinoma, gastric cancer, colon cancer, neuroblastoma including aggressive neuroblastoma and from an EphB6-deficient cancer listed in FIG. 1.

43. The method of claim 11, wherein the SRC kinase inhibitor is selected from dasatinib, bosutinib (SKI-606), saracatinib (AZD530), SU6656, KX2-391 and/or posatinib (AP24534), PPI, PP2, Quercetin and/or pharmaceutically acceptable salts, solvates, and/or hydrates thereof.

44. The method of claim 11 , wherein the MET kinase inhibitor is selected from tivantinib (ARQ197), K252a, SU11274, AM7, PHA-665752, PF-2341066, foretinib, SGX523, MP470, crizotinib, cabozantinib, and/or pharmaceutically acceptable salts, solvates, and/or hydrates thereof.
